(19) United States
(12) Patent Application Publication
(10) Pub. No.: US 2008/0195170 A1
(43) Pub. Date: Aug. 14, 2008

(54) MEDICAL DEVICE WITH EXTENDED OR MULTIPLE RESERVOIRS

(75) Inventor: Soheil Asgari, Wiesbaden (DE)

Correspondence Address: DORSEY & WHITNEY LLP;INTELLECTUAL PROPERTY DEPARTMENT, 250 PARK AVENUE, NEW YORK, NY, 10177, US

(73) Assignee: CINVENTION AG, Wiesbaden (DE)

(21) Appl. No.: 12/030,304

(22) Filed: Feb. 13, 2008

Related U.S. Application Data
(60) Provisional application No. 60/889,708, filed on Feb. 13, 2007.

(51) Int. Cl. A61N 1/00; A61F 2/00

(52) U.S. Cl. 607/36; 424/423

(57) ABSTRACT

An exemplary embodiment of a process can be provided for a manufacture of an implantable medical device or a part thereof. For example, at least one metallic layer can be deposited on a three-dimensional template of the device and at least partially removing the template. Implants can be produced, e.g., which may have relatively large reservoirs for including an active ingredient, such as a pharmacologically, therapeutically or biologically active agent, a diagnostically active agent, a marker, an absorptive agent, for eluting in-vivo.

### CROSS-REFERENCE TO RELATED APPLICATION(S)

The present invention claims priority from U.S. Provisional Application Ser. No. 60/889,708 filed Feb. 13, 2007, the entire disclosure of which is incorporated herein by reference.

### FIELD OF THE PRESENT INVENTION

The present invention relates to a process for the manufacture of an implantable medical device or a part thereof wherein at least one metallic layer is deposited on a three-dimensional template of the device and at least partially removing the template. For example, implants may be produced, which have relatively large reservoirs for including an active ingredient, such as a pharmacologically, therapeutically or biologically active agent, a diagnostically active agent, a marker, an absorptive agent, for eluting in-vivo.

### BACKGROUND INFORMATION

Implants are widely used as short-term or long-term devices to be implanted into the human body in different fields of applications, such as orthopedic, cardiovascular or surgical reconstructive treatments. Typically, implants can be made of solid materials, either polymers, ceramics or metals. To enable drug-delivery, implants have also been produced with porous structures or by using porous materials, whereas a drug may be included for in-vivo release.

Since the amount of drug available in porous structures is limited, larger drug reservoirs in medical implant structures have been envisaged to increase the drug loading volume. For example, European patent application EP 1 466 634 A1 describes a stent design with drug reservoirs by introducing through-holes capable of being filled with a drug either in metallic or polymeric stents by laser cutting, etching, drilling or sawing or the like.

PCT patent publication WO 96/26682 describes a hollow stent made of a tubular wire, wherein a pharmacological agent may be included inside the lumen of the wire for release through a plurality of openings in the tubular wire.

Japanese patent application JP 2005-328893 A describes a stent structure with hollow sections for housing a medicament which may be released through small holes. The hollow structure is produced by a sequence of several deposition and etching procedures.

There may be an increasing demand for functional implants that provide a larger available space unit for storage and for delivery of biologically active, pharmacologically active, therapeutically active, diagnostic or absorptive agents into the living organism. Furthermore, there is an increasing demand of using multiple agents or agents that must be available in higher amounts than currently applicable. One exemplary disadvantage of conventional solutions is that the overall space or free volume available for the uptake of active ingredients in the implant is typically limited. Particularly with coated implants, e.g., the amount of active agents may be limited either due to the use of polymers or other carriers containing one or more agents, or due to the limited volume that can be provided by the pore system.

### SUMMARY OF EXEMPLARY EMBODIMENTS OF PRESENT INVENTION

It is one of the objects of the exemplary embodiments of the present invention to provide an implant capable of releasing active ingredients, such as a drug or a marker or a diagnostic agent etc., and a method for its manufacture. A further object of the exemplary embodiments of the present invention is to provide an implant design that facilitates an increase of the effective volume of space usable as a reservoir for active ingredients. Another object of the exemplary embodiments of the present invention is to provide an implant design that facilitates providing at least two different lumens usable as reservoirs for active ingredients.

A further object of the exemplary embodiments of the present invention is to provide an implant that can be used as a device for controlled release of active ingredients. Another object of the exemplary embodiments of the present invention is to provide multifunctional implants which can be modified in their material properties, particularly the physical, chemical and biologic properties, e.g. biodegradability, x-ray and MRI visibility or mechanical strength. Another object of the exemplary embodiments of the present invention is to provide a cardiovascular implant that comprises a hollow, interconnected tubular network as a reservoir for active ingredients. A further object of the exemplary embodiments of the present invention is to provide orthopedic, traumatologic or surgical devices, particularly plates, screws, nails, bone grafts, adhesive implants, and the like, that comprise a hollow space as a reservoir for active ingredients. Another object of the exemplary embodiments of the present invention is to provide an implantable device for use as wound dressings or gynecologic implants. A further object of the present invention is to provide a simple and cost-effective, flexible process for the manufacturing of such medical implants as described above, having at least one hollow space or lumen which can be used as a reservoir for active ingredients.

According to one exemplary embodiment of the present invention a process for the manufacture of an implantable medical device or a part thereof can be provided. Using such exemplary process, it is possible to:

i) provide a three-dimensional template of the device or part thereof,

ii) deposit at least one metallic layer covering the template, and

iii) at least partially remove the template.

In another exemplary embodiment of the present invention, it is possible to create at least one hollow space, other than a pore, within a metal-based structure defined by the metallic layer.

In one further exemplary embodiment of the present invention, the metallic layer substantially can completely cover the template. The deposition of the metallic layer may preferably be performed in a one step operation. In such embodiments, the metallic layer may be porous to allow removal of the template, or at least one opening may be provided in the metallic layer before removal of the template. In another exemplary embodiment of the present invention, the metallic layer may cover the template partially. In still another exemplary embodiment of the present invention, the metallic layer may be deposited by a conventional deposition method, such as at least one of CVD, PVD, electroplating, electro deposition, electroless plating, sol/gel precipitation, or the like.

In still further exemplary embodiment of the present invention, the hollow space within the metallic implant structure can be filled at least partly with an active ingredient. In one further at least one, at least one opening or a plurality of openings may be provided in the metallic layer after removal of the template. Such openings may be provided to allow for a release of an active ingredient included in the hollow space within the metallic implant structure, or to absorb a compound by provision of an absorptive agent included in the hollow space.

The templates may be of a polymeric material, which can be removed in-vivo or ex-vivo. Removal of the template in-vivo may be done e.g. by using biodegradable materials for the template structure or parts thereof, which materials are dissolvable or degradable in the presence of physiologic fluids, or which can be metabolized after implantation of the device by the organism. Ex-vivo removal of the template may be accomplished e.g. by dissolving the template with suitable solvents from the remaining hollow metallic structure of the implant, or by degrading the template thermally, e.g. pyrolysis or evaporation, or by applying mechanically induced destruction, such as lithotripsy, ultrasound and the like, or inducing bimetallic corrosion.

In exemplary embodiments of the present invention, the metallic layer covering the template may be of any suitable metal or metal alloy, preferably of a biocompatible metallic material.

In certain exemplary embodiments of the present invention, it may be preferable to use biodegradable metallic layers. Typical examples for biodegradable metallic materials can include Mg or Zn, or an alloy comprising at least one of Mg, Ca, Fe, Zn, Al, W, Ln, Si, or Y.

In a further exemplary embodiments of the present invention, an implant may be provided, producible by the exemplary methods as described above. The implant may include at least one active ingredient, such as a pharmacologically active agent, a diagnostically active agent, a marker, an absorptive agent as described herein below, or any combination thereof. In certain exemplary embodiments of the present invention, the implantable medical device may further include the active ingredients in at least one of the hollow spaces or lumens created by removing the template. The active ingredient may be configured to be released from the lumen of the implant, for example in-vivo into a vessel or other parts of the body, or ex-vivo. The implant may be, for example, a vascular endoprosthesis, an intraluminal endoprosthesis, a stent, a coronary stent, a peripheral stent, a surgical or orthopedic implant, an implantable orthopedic fixation aid, an orthopedic bone prosthesis or joint prosthesis, a bone substitute or a vertebral substitute in the thoracic or lumbar region of the spinal column; an artificial heart or a part thereof, an artificial heart valve, a heart pacemaker casing or electrode, a subcutaneous and/or intramuscular implant, an implantable drug-delivery device, a microchip, or implantable surgical needles, screws, nails, clips, or staples.

In another exemplary embodiment of the present invention, at least one active ingredient may be included in an in-vivo biodegradable template material, for being releasable in-vivo through at least one opening in the metallic layer of the implant.

These and other objects, features and advantages of the present invention will become apparent upon reading the following detailed description of embodiments of the present invention.

### DETAILED DESCRIPTION OF EXEMPLARY EMBODIMENTS

The terms “template”, “three-dimensional template” or “template structure”, as used herein can mean, but in no way limited to include, e.g., a three-dimensional structure or model of the intended implant or part thereof to be produced, which may serve as an intermediate carrier for enabling the production of a metallic implant structure by applying or depositing a metallic layer surrounding or covering at least a part of the template, such that after removal of the template a metallic implant structure (or part thereof) remains, that comprises at least one hollow space at a position in the metallic implant structure, which was occupied by the template during manufacture of the implant. For example, the template typically has a complex form, other than a particle or particulate form, that essentially defines the shape or form of the metallic implant structure. For example, the implant or part thereof may have a shape of a sandwiched, optionally multilayered sheet or tube, wherein the sandwich may comprise a tube or a sheet of the desired implant material defining the outer and the inner shell, whereby the core of the sandwich can comprise any removable or degradable material. In this context, the core of the sandwich is referred to as a template for generating the inner hollow space or respective reservoir. Removing the templates may result in the formation of a lumen within the implant or part thereof.

The term “biodegradable” as used herein can include, but in no way limited to include, e.g., any material which can be removed in-vivo, e.g. by biocorrosion or biodegradation. Thus, any material, e.g., a metal or organic polymer that can be degraded, absorbed, metabolized, or which is resorbable in the human or animal body may be used either for a biodegradable metallic layer or as a biodegradable template in the exemplary embodiments of the present invention. Also, as used in this description, the terms “biodegradable”, “bioabsorbable”, “resorbable”, and “biocorrodible” can include, but in no way limited to include, e.g., materials that are broken down and may be gradually absorbed or eliminated by the body in-vivo, regardless whether these processes are due to hydrolysis, metabolic processes, bulk or surface erosion.

The term “metallic layer” as used herein can include, but in no way limited to include, e.g., inorganic materials, such as metals and alloys, metal-based compounds or composites including metal atoms or metal ions, such as e.g. ceramics, oxides, nitrides, carbides, silica, zeolite, etc.

The terms “active ingredient” or “active agent” as used herein can include, but in no way limited to include, e.g., any material or substance which may be used to add a further function to the implantable medical device. Examples of active ingredients include biologically, therapeutically or pharmacologically active agents, such as drugs or medicaments, diagnostic agents, such as markers, or absorptive agents. The active ingredients may be a part of the template or the metallic layer, such as incorporated into the implant or being coated on at least a part of the implant. Biologically or therapeutically active agents comprise substances being capable of providing a direct or indirect therapeutic, physiologic and/or pharmacologic effect in a human or animal organism. The therapeutically or pharmacologically active agent may include a drug, pro-drug or even a targeting group or a drug comprising a targeting group. Examples for biologically active ingredients may include living cells or tissue, microorganisms, such as bacteria, fungi, algae, virus; enzymes, vectors, targeting-groups etc. An “active ingredient” according to the exemplary embodiment of the present invention may further include a material or substance which may be activated physically, e.g. by radiation, or chemically, e.g. by a metabolic process.

Without wishing to be bound to any particular theory, it has been determined that with the processes of embodiments of the present invention implants may be produced which may comprise substantially larger volumes of space which may be used as a reservoir for active ingredients. Particularly, the exemplary process facilitates a creation of at least one hollow space within a metallic structure defined by the metallic layer, which may be used e.g. as a reservoir for a specific amount of drug to be released after implantation into the body.

Exemplary Template

Using the process according to the exemplary embodiments of the present invention, implants may be manufactured e.g. in one seamless part or with seams from multiple parts. In case of an implant formed from a plurality of parts, a plurality of the same or different templates may be used, and the final implant may be manufactured from the metallized parts after removal of the templates, or by combining metallized templates and thereafter removing the template(s). Also, e.g. a sheet-like template may be further processed after metallization to obtain the implant structure, e.g. by rolling into a cylindrical shape, before or after removal of the template.

The templates may be manufactured in the desired shape using conventional implant manufacturing techniques. For example, suitable manufacturing methods may include, but are not limited to, laser cutting, chemical etching, weaving of fibers, stamping of tubes, stamping of flat sheets, rolling of sheets into cylindrical shapes and, as a further option, e.g. welding or gluing of sheets, fibers or other shapes of template material. Other manufacturing techniques can include electrode discharge machining or molding the inventive implant with the desired design. A further option may be to weld or glue individual sections of the template together. Bulk materials may be structured into templates, for example, by folding, embossing, punching, pressing, extruding, gathering, injection molding, and the like. In this way, certain structures of a regular or irregular type may be provided for use as a template according to exemplary embodiments of this invention. Other methods to form a template may include shaping of materials in liquid, pulpy or pasty form, for example, extruding, slip casting, or molding, and hardening the three dimensional template shape, if desired. Other conventional methods to provide templates may include wet or dry spinning methods, electro-spinning and the like, or knitting, weaving and any other known method to produce woven or non-woven articles or forms of regular or irregular shape. For cylindrical or tube-like implant designs, templates may be provided as sheets, foils or tubes, such as sandwiched tubes or sandwiched sheets. The template may be provided in a substantially net shape of the desired implant design.

Materials suitable for providing a template in the exemplary embodiments of the present invention can include any materials, substances, compounds, or mixtures thereof that can be metallized by conventional methods suitable for depositing a metallic layer and that can be removed by physical, chemical or mechanical means, preferably substantially without substantially affecting the metal phase of the metallized templates.

Such exemplary materials can include, for example, organic polymer materials that can be thermally degradable, vaporizable, i.e. they may be substantially completely decomposed or evaporated under the conditions of elevated temperatures, or which may be dissolved by suitable solvents. Examples of template materials may include, for example, polymers, oligomers, or pre-polymerized forms as well as all substances which may be synthesized to pre-polymeric, partially polymerized or polymeric materials or which are already present as such materials, including polymer composites, thermosets, thermoplastics, synthetic rubbers, extrudable polymers, injection molding polymers, moldable polymers, spinable, weaveable and knittable polymeric structures, oligomers or pre-polymerizes forms and the like or mixtures thereof. Further examples can include poly(meth)acrylate, unsaturated polyester, saturated polyester, polyolefines, such as polyethylene, polypropylene, polybutylene, alkyd resins, epoxy-polymers or resins, polyamide, polyimide, polyetherimide, polyamideimide, polyesterimide, polyester amide imide, polyurethane, polycarbonate, polystyrene, polyphenol, polyvinyl ester, polysilicone, polyacetal, cellulosic acetate, polyvinylchloride, polyvinyl acetate, polyvinyl alcohol, polysulfone, polyphenylsulfone, polyethersulfone, polyketone, polyetherketone, polybenzimidazole, polybenzoxazole, polybenzthiazole, polyfluorocarbons, polyphenylene ether, polyarylate, cyanatoester-polymers, and mixtures or copolymers of any of the foregoing.

In addition, exemplary templates can be made from materials selected from poly(meth)acrylates based on mono(meth)acrylate, di(meth)acrylate, tri(meth)acrylate, tetra-acrylate and pentaacrylate; as well as mixtures, copolymers and combinations of any of the foregoing. Examples for polyacrylates may be polyisobornylacrylate, polyisobornylmethacrylate, polyethoxyethoxyethylacrylate, poly-2-carboxyethylacrylate, polyethylhexylacrylate, poly-2-hydroxyethylacrylate, poly-2-phenoxylethylacrylate, poly-2-phenoxyethylmethacrylate, poly-2-ethylbutylmethacrylate, poly-9-anthracenylmethyl methacrylate, poly-4-chlorophenylacrylate, polycyclohexylacrylate, polydicyclopentenyloxyethylacrylate, poly-2-(N,N-diethylamino)ethylmethacrylate, poly-dimethylaminoeopentylacrylate, poly-caprolactone 2-(methacryloxy)ethylester, or polyfurfurylmethacrylate, poly(ethylene glycol)methacrylate, polyacrylic acid and poly(propylene glycol)methacrylate.

Suitable polyacrylates may also comprise aliphatic unsaturated organic compounds, such as e.g. polyacrylamide and unsaturated polyesters from condensation reactions of unsaturated dicarboxylic acids and diols, as well as vinyl-derivatives, or compounds having terminal double bonds. Specific examples include N-vinylpyrollidone, styrene, vinyl-naphthalene or vinylphtalimide. Also suitable may be methacrylamid-derivatives, such as N-alkyl- or N-alkylen-substituted or unsubstituted (meth)acrylamide, e.g. acrylamide, methacrylamide, N-methacrylamide, N-methylmethacrylamide, N-ethylacrylamide, N,N-dimethylacrylamide, N,N-dimethylmethacrylamide, N,N-diethylacrylamide, N-ethylmethacrylamide, N-methyl-N-ethylacrylamide, N-isopropylacrylamide, N-n-propylacrylamide, N-isopropylmethacrylamide, N-n-propylmethacrylamide, N-acryloyloylpyrrolidine, N-methacryloylpyrrolidine, N-acryloylpiperidine, N-methacryloylpiperidine, N-acryloylhexahydroazepine, N-acryloylmorpholine or N-methacryloylmorpholine.

Further suitable template materials may include unsaturated and saturated polyesters, including alkyd resins. The polyesters may contain polymeric chains, a varying number of saturated or aromatic dibasic acids and anhydrides. Furthermore, epoxy resins, which may be used as monomers, oligomers or polymers are suitable, particularly those which can comprise one or several oxirane rings, one aliphatic, aromatic or mixed aliphatic-aromatic molecular structural element, or exclusively non-benzoid structures, i.e., aliphatic or cyclophatic structures with our without substituents, such as halogen, ester groups, ether groups, sulfonate groups, siloxane groups, nitro groups, or phosphate groups, or any combination thereof. Specific examples include epoxy resins of the glycidyl-epoxy type, for example equipped with the diglycidyl groups of bisphenol A, or amino derivatized epoxy resins, such as tetraglycidyl diaminodiphenyl methane, triglycidyl-p-aminophenol, triglycidyl-m-maminophenole, or triglycidyl aminocresole and their isomers, phenol derivatized epoxy resins like, for example, epoxy resins of bisphenol A, bisphenol F, bisphenol S, phenol-novolac, cresole-novolac or resorcinole as well as alicyclic epoxy resins. Furthermore, halogenated epoxy resins, glycidyl ethers of polyhydric phenols, diglycidylether of bisphenol A, glycidylethers of phenole-formaldehyde-novolac resins and resorcinole diglycidylether, as well as further epoxy resins as described in U.S. Pat. No. 3,018,261, may be used.

In accordance with certain exemplary embodiments of the present invention, the selection of the template material is likely not restricted to the examples mentioned above. For example, mixtures of epoxy resins from two or several components as described above may also be selected, as well as mono-epoxy components. The epoxy resins in the exemplary embodiments can also include resins which may be crosslinked via UV radiation, as well as cycloaliphatic resins.

Further suitable polymers can include polyamides, such as aliphatic or aromatic polyamides and aramides (Nomex®), and their derivatives, nylon-6-(polycaprolactam), nylon 6/6 (polyhexamethyleneadipamide), nylon 6/10, nylon 6/12, nylon 6/T (polyhexamethylene terephthalamide), nylon 7 (polyenanthamide), nylon 8 (polycapryllactam), nylon 9 (polypelargonamide), nylon 10, nylon 11, nylon 12, nylon 55, nylon XD6 (poly metha-xylylene adipamide), nylon 6/I, and poly-alanine.

In a further exemplary embodiment, metal phosphinates or polymetal phosphinates as well as inorganic metal-containing polymers or organic metal-containing polymers like, for example, metallodendrimers, metallocenyl polymers, carbosilanes, polyynes, noble metal alkynyl polymers, metalloporphyrine polymers, metallocenophanes, metallocenylsilane-carbosilane copolymers as mono, diblock, triblock or multiblock copolymers may be used, as well as poly(metallocenyldimethylsilane) compounds, carbothiametallocenophanes, poly(carbothiametallocenes) and the like, wherein this list of compounds is not exclusive and any combinations thereof may be used.

In a certain exemplary embodiment, the template material may be selected from saturated or unsaturated polyparaphenylene-vinylene, polyparaphenylene, polyaniline, polythiophene, poly(ethylenedioxythiophene), polydialkylfluorene, polyazine, polyfurane, polypyrrole, polyselenophene, poly-p-phenylene sulfide, polyacetylene, oligomers or polymers thereof or any combinations and mixtures thereof with other oligomers or polymers or copolymers.

In some exemplary embodiments, the use of biodegradable polymers, such as collagen, albumin, gelatin, hyaluronic acid, starch, celluloses, such as methylcellulose, hydroxypropyl cellulose, hydroxypropyl methylcellulose, carboxymethylcellulose phthalate; casein, dextranes, polysaccharides, fibrinogen, poly(D,L-lactides), poly(D,L-lactide coglycolides), polyglycolides, polyhydroxybutylates, polyalkyl carbonates, polyorthoesters, polyesters, polyhydroxyvaleric acid, polydioxanones, polyethylene terephthalates, polymaleate acid, polytartronic acid, polyanhydrides, polyphosphazenes, polyamino acids; polyethylene vinyl acetate, silicones; poly(ester urethanes), poly(ether urethanes), poly(ester ureas), polyethers, such as polyethylene oxide, polypropylene oxide, pluronics, polytetramethylene glycol; polyvinylpyrrolidone, poly(vinyl acetate phthalate), shellac, and combinations of these homopolymers or copolymers may be a preferred option. In such embodiments, the polymeric template may be, for example, impregnated with at least one active ingredient, which can be eluted in-vivo before or during degradation of the template by metabolic processes in the body.

In certain exemplary embodiments, it may also be desirable to make the template from inorganic materials, such as metals, alloys, metal oxides, metal carbides, metal nitrides, metal oxynitrides, metal carbonitrides, metal oxycarbides, metal oxynitrides, metal oxycarbonitrides, or inorganic metal salts, such as salts from alkaline and/or alkaline earth metals and/or transition metals, for example, alkaline or alkaline earth metal carbonates, -sulfates, -sulfites, -nitrates, nitrites, -phosphates, -phosphites, -halides, -sulfides, -oxides, as well as mixtures thereof; or organic metal salts, such as alkaline or alkaline earth and/or transition metal salts, for example, their formiates, acetates, propionates, malates, maleates, oxalates, tartrates, citrates, benzoates, salicylates, phtalates, stearates, phenolates, sulfonates, and amines; as well as mixtures thereof.

In these exemplary embodiments the template material should be selected to be removable substantially without affecting the metallic layer of the implant. For example, the template may be made from biodegradable or dissolvable inorganic materials, whereas the metallic layer forming the implant structure may be made from a substantially non-biodegradable or non-dissolvable metal or alloy. Another example can comprise a metallic template that can be easily degraded by induced corrosion, while the metallic layer forming the implant structure may be selected from materials being corrosion resistant under the selected conditions of induced corrosion.

An induced corrosion can, for example, be achieved by providing, as the template material, alloy compositions that can be degraded at least partially under artificially created corrosive conditions. Such conditions may be created for example by electrochemical methods, or the use of acids to dissolve the metallic template underneath the acid-resistant metallic layer deposited thereon. Another example may include the combination of different metals or metal alloys with a difference in electronegativity for use as the template and the metallic layer, which can result in a selective corrosion of the less noble metal compounds of the template by providing an electrolyte that bridges both metals, preferably by immersing them into the electrolyte. Such a corrosion can also be induced by directly coupling both different metals physically, which is typically the case when depositing a more electropositive metallic layer onto a more electronegative template material. This exemplary combination can result in a bimetallic corrosion, wherein the electronegative metal template is corroded while the electropositive metallic layer remains substantially unaffected. Induced corrosion may be accomplished ex-vivo or in-vivo in the presence of physiologic fluids. For example, a template including biodegradable materials such as magnesium-based alloys may be corroded in-vivo under formation of hydroxyl apatite, and the metallic layer of a non-degradable material remains in the body and encloses the lumen or reservoir determined by the degraded template.

An exemplary embodiment for bimetallic corrosion induced manufacture of reservoir implants can include the use of e.g. a template including magnesium, zinc, aluminum or an alloy comprising these metals, or a biodegradable metallic material as further described below, where a metallic layer comprising more noble metals, such as gold, platinum, titanium or copper is deposited.

In some exemplary embodiments of the present invention, the metals for the template and/or the metallic layer may be selected from main group metals of the periodic system, transition metals, such as copper, gold and silver, titanium, zirconium, hafnium, vanadium, niobium, tantalum, chromium, molybdenum, tungsten, manganese, rhenium, iron, cobalt, nickel, ruthenium, rhodium, palladium, osmium, iridium or platinum, or from rare earth metals or any oxide, carbide, nitride, oxynitride, carbonitride, oxycarbide, oxynitride, oxycarbonitride, any alloy thereof or any mixture thereof.

The metal based compounds preferably used in some exemplary embodiments are, without excluding others, e.g.—iron, copper, cobalt, nickel, manganese or mixtures thereof, e.g. iron-platinum-mixtures, or as an example for magnetic metal oxides iron oxides and ferrites.

In certain exemplary embodiments, the templates may be made from composites comprising metal and non-metal materials, for example to increase mechanical and form stability of the template during processing or to facilitate the metallization step. In other exemplary embodiments, particularly if the removal of the template material occurs in-vivo within the living organism, the template material may be selected specifically from biocompatible and/or bio-degradable polymers, composites, or metals or any mixture thereof.

In further exemplary embodiments of the present invention, biodegradable metallic template materials may be selected from biodegradable or biocorrosive metals or alloys based on at least one of magnesium or zinc, or an alloy comprising at least one of Mg, Ca, Fe, Zn, Al, W, Ln, Si, or Y. Furthermore, the material may be substantially completely or at least partially degradable in-vivo. Examples for suitable biodegradable alloys comprise magnesium alloys comprising more than 90% of Mg, about 4-5% of Y, and about 1.5-4% of other rare earth metals, such as neodymium and optionally minor amounts of Zr; or biocorrosive alloys comprising as a major component tungsten, rhenium, osmium or molybdenum, for example alloyed with cerium, an actinide, iron, tantalum, platinum, gold, gadolinium, yttrium or scandium.

Specific examples can further include alloys comprising:
(i) 10-98 wt.-%, such as 35-75 wt.-% of Mg, and 0-70 wt.-%, such as 30-40% of Li and 0-12 wt.-% of other metals, or
(ii) 60-99 wt.-% of Fe, 0.05-6 wt.-% Cr, 0.05-7 wt.-% Ni and up to 10 wt.-% of other metals; or
(iii) 60-96 wt.-% Fe, 1-10 wt.-% Cr, 0.05-3 wt.-% Ni and 0-15 wt.-% of other metals,
   whereas the individual weight ranges can be selected to always add up to 100 wt.-% in total for each alloy. In addition, both metal-based and non-metallic biodegradable materials may be combined to provide a suitable template.

In other exemplary embodiments, a template may be produced from an aerogel or xerogel in a sol/gel process as described below, and the gel may be coated with a metallic layer with appropriate metallization methods, such as wet chemical metallization or PVD methods explained below.

The templates may be any articles that essentially provide the three-dimensional shape of an implant. Exemplary shapes may include meshes, lattices, either planar or in any three-dimensional regular or irregular form, screw- and nail-like shapes, plates and the like. Also preferred are helically wounded tubes, lattices, wrapped meshes and the like.

Exemplary Metallization

In the process according to the exemplary embodiments of the present invention, the template may be metallized, i.e. at least one metallic layer can be deposited on the template. In addition, a plurality of metallic layers may be deposited, optionally layers of different materials, such as metals or alloys. Metallization may result in forming of a metal phase or a metal-based material on the outer and inner surfaces of the template. The metal phase or metallic layer may cover the template substantially completely, or at least in part, and it may be continuous, non-porous or porous. Before depositing the metallic layer, the template may be provided in dry or wet form, and may optionally be pretreated, e.g. coated or wetted with a primer, or any other suitable material to facilitate metallization of the template.

In certain exemplary embodiments, the metallic layer may be produced by depositing at least one of a metal or alloy of e.g. main group metals of the periodic system, transition metals, such as copper, gold and silver, titanium, zirconium, hafnium, vanadium, niobium, tantalum, chromium, molybdenum, tungsten, manganese, rhenium, iron, cobalt, nickel, ruthenium, rhodium, palladium, osmium, iridium or platinum, or from rare earth metals or any oxide, carbide, nitride, oxynitride, carbonitride, oxycarbide, oxynitride, oxycarbonitride, any alloy or any mixture thereof, further including biodegradable or biocorrosive metallic materials, such as those based on at least one of magnesium or zinc, or an alloy comprising at least one of Mg, Ca, Fe, Zn, Al, W, Ln, Si, or Y. The material used for the metallic layer may be substantially completely or at least partially degradable in-vivo. Examples for suitable biodegradable alloys comprise magnesium alloys comprising more than 90% of Mg, about 4-5% of Y, and about 1.5-4% of other rare earth metals, such as neodymium and optionally minor amounts of Zr; or biocorrosive alloys comprising as a major component tungsten, rhenium, osmium or molybdenum, for example alloyed with cerium, an actinide, iron, tantalum, platinum, gold, gadolinium, yttrium or scandium.

Specific examples further include alloys comprising:
(i) 10-98 wt.-%, such as 35-75 wt.-% of Mg, and 0-70 wt.-%, such as 30-40% of Li and 0-12 wt.-% of other metals, or
(ii) 60-99 wt.-% of Fe, 0.05-6 wt.-% Cr, 0.05-7 wt.-% Ni and up to 10 wt.-% of other metals; or
(iii) 60-96 wt.-% Fe, 1-10 wt.-% Cr, 0.05-3 wt.-% Ni and 0-15 wt.-% of other metals,
   whereas the individual weight ranges can be selected to always add up to 100 wt.-% in total for each alloy. Further, both metal-based and non-metallic biodegradable materials may be combined in a metallic layer.

The deposition of the metallic layer onto the discrete template may be carried out by any suitable conventional technique, for example PVD methods, such as vapor deposition or sputter techniques, or by CVD (chemical vapor deposition) procedures, such as thermal CVD or Plasma Enhanced CVD (PECVD), ALD (Atomic Layer Deposition), such as thermal ALD, Plasma Assisted ALD or Plasma Enhanced ALD, electrolytic methods such as electroplating, or electroless, wet-chemical metallization or plating, respectively. The methods listed here are not exclusively selected and can be substituted.

Exemplary Plating

In certain exemplary embodiments of the present invention, the template may be metallized in a liquid plating process with metal-containing solutions. Liquid plating processes can include, for example, electroplating or electrodeposition, and electroless plating.

Electroplating of the template may be achieved by passing an electrical current through a solution or dispersion containing dissolved metal ions and the template to be plated. Typically, this may involve an aqueous plating bath comprising the chemical solution which contains an ionic form of at least one metal, a consumable, sacrificial anode which comprises the metal being plated, or an inconsumable anode which consists of, e.g. carbon, platinum, titanium, lead or steel, and a cathode, which may be the template to be coated, where electrons are supplied to produce a metal film. Electroplating can be used to deposit a single metallic element or alloys, such as, for example, Ni, Au, Ag, Cu, Fe, Co, Fe—Ni, C—Ni, Ni—Ti, Co—Cr, Ru, Pt, Cr, Pd, Mg, Zn, Sn, Pb, Cd, brass or solder.

The plating bath may include additives, e.g., cyanides of other metals (e.g., potassium cyanide) in addition to cyanide salts of the metal to be deposited. Excess cyanides can facilitate anode corrosion, may help to maintain a constant metal ion level and contribute to the electrical conductivity. Additionally, non-metal chemicals, such as carbonates and phosphates may be added to increase conductivity. The process can be regulated by controlling a variety of parameters, including the voltage and amperage, temperature, residence times, and the purity of bath solutions.

The electrodeposition of a metallic layer on a template according to an exemplary embodiment of the process of the present invention may also be done using a non-aqueous electrolyte. Non-aqueous electrolytes can comprise molten salts, inorganic or organic solvents. Molten salts include, e.g., KCl/NaCl or Li₂CO₃/K₂CO₃, inorganic solvents include, e.g., liquefied gases, such as NH₃ or SO₂, and organic solvents include, e.g., methanol, ethanol, propanol, or diethylether. In certain exemplary embodiments, elements, such as Ti and Al may be deposited from organic electrolytes, while other metals, such as Mg, Nb, Ta, and W may be plated from molten salt electrolytes, e.g. at temperatures of 700° C. and above, depending on the electrolyte used. In such a process, inorganic essentially temperature resistant templates may be used.

Before the template is plated, it may be pretreated as desired, such as by cleansing or pre-treating with other chemicals, that will facilitate deposition, enhance the deposition speed, or increase adhesion with the plating metal. In certain exemplary embodiments, surface treatment and plating operations may have three basic steps: Surface cleaning or preparation, which may include employing suitable agents, such as solvents, alkaline cleaners, acid cleaners, abrasive materials and/or water, optionally followed by a surface modification which may include a change in surface attributes, such as application of a primer or surface modifier, at least one layer and/or hardening, and finally rinsing or other work-piece finishing operations to obtain the final product.

Non-metallic or nonconductive templates may first be processed through a pre-plate cycle, during which a metallic coating may be deposited, for example by an electroless plating process, to render the template conductive, before an electroplating process is applied.

In other exemplary embodiments, electroless plating may be used for metallizing the template. Electroless plating may be used for depositing metals on metallic and non-metallic templates, typically in a wet-chemical process from a plating bath, i.e. without the use of electrical current. Electroless plating involves a chemical reduction of at least one metal ion onto a surface. The surface may be autocatalytic to this process, as may be the case with metallic templates, and the deposition of the metallic layer is typically induced by a chemical reducing agent in solution. Sufficient agitation of the plating bath may be favorable to ensure a uniform concentration of metal ions and reducing agents at all points of the surface of the template.

A variety of additives may be used in electroless or electroplating methods, such as stabilizers, such as chelating agents, acids or bases for adjusting the pH, or buffers. Chelating and/or complexing agents that hold the metals in solution may be used in plating baths for electroless or electroplating. Common chelating agents can, e.g., include ethylenediaminetetraacetic acid (EDTA), citrates, oxalates, cyanides, and 1,2 diaminocyclohexanetetraacetic acid (DCTA). Deposition rates may be controlled by the amount of reducing agent present and/or the type of chelating agent used.

Since electroless plating is a chemical process, metal deposition typically proceeds with excellent uniformity over the entire surface of the template, which may be preferred in certain exemplary embodiments of the present invention.

In the case of metallizing non-metallic templates, in an exemplary conventionally used procedure the surface of the template may be made auto-catalytic in a pre-plate cycle, before the electroless plating process, by conventional measures, for example through the adsorption of a catalyst onto the surface of the template, or by application of a coating comprising catalytic materials. An exemplary pre-plate cycle for a template to be metallized may for example comprise etching, neutralizing, catalyzing and acceleration. The etch bath may consist of an acidic solution, such as a highly concentrated solution of chromic and sulfuric acid, which may oxidize selective areas on the template. The small holes produced by the oxidizing action may function as absorbing sites that hold small metallic particles that may serve as activators for electroless plating. The hole size may influence adhesion and other physical properties. The neutralizing bath may contain e.g. mild acids or alkaline solutions or other suitable substances that chemically neutralize the acids from the etching bath. In a catalyzing step, a catalytic film of, e.g. a tin-palladium catalyst, can be put on the oxidized surface to prepare for electroless metal deposition, and finally the accelerator bath may be used to remove all the chemical that remain after the catalyzing procedure, before a metallic film or layer is deposited on the template using electroless plating.

Reducing agents for electroless plating may include, for example, NaH₂PO₂, dimethyl amino borane (DMAB), sodium tetrahydroborate (SBH), formaldehyde or glyoxylic acid.

The above described exemplary embodiments of plating methods can typically produce continuous, non-porous coatings. Openings or discontinuities in the metallic layer may be provided during plating, e.g. by conventional masking techniques, such as masking certain areas of the template surface with a material on which the deposited metallic layer does not adhere. Such openings or discontinuities may be used for removing the template from inside the metallic layer coating, and/or for eluting active ingredients from the reservoir inside the implant.

### Sol-Gel

To obtain a porous surface of the metallic layer or a substantially completely porous metallic layer, deposition of a metallic layer may be performed by using conventional sol/gel techniques. Such exemplary techniques may, depending on the materials and additives such as pore-formers, removable fillers or the like used, lead to porous metallic layers which allow a fluid communication between the outer environment of the implant and the template or reservoir inside the metallic layers. For example, the particle size of the sol/gel components or additives used to produce the metallic layer may determine the porosity and pore sizes in the metallic layer. For example, in certain exemplary embodiments of the present invention, an aerogel or xerogel metal-based layer may be deposited on the template by sol/gel technology. Deposition of the metallic layer may be achieved e.g. by using a sol of a metal or metal compound, such as a metal salt, the sol being applied to the template by suitable methods, such as dipping, spraying etc. and the deposition step may then include an induced precipitation of the metallic or metal-based materials from the sol onto the template. The precipitation or formation of a solid aerogel or xerogel may be conventionally induced by drying, ageing, crosslinking, hydrolysis or the like.

Sols can, e.g., be used to modify the pore sizes and the degree of porosity of the metallic layer, if desired. Exemplary sols may be based on inorganic metal salts, such as salts from alkaline and/or alkaline earth metals, for example alkaline or alkaline earth metal carbonates, -sulfates, -sulfites, -nitrates, nitrites, -phosphates, -phosphites, -halides, -sulfides, -oxides, as well as mixtures thereof. Further suitable salts include organic metal salts, e.g. alkaline or alkaline earth and/or transition metal salts, such as their formiates, acetates, propionates, malates, maleates, oxalates, tartrates, citrates, benzoates, salicylates, phtalates, stearates, phenolates, sulfonates, and amines, as well as any mixture thereof.

The sols may be prepared from any type of sol/gel forming components in a conventional manner. Those having ordinary skill in the art—depending on the desired properties and requirements of the material to be produced—can select the suitable components/sols for coating the templates based on his professional knowledge.

For example, the sol/gel forming components in the exemplary embodiment of the process according to the present invention may be selected from alkoxides, oxides, acetates, nitrates of various metals, e.g. silicon, aluminum, boron, magnesium, zirconium, titanium, alkaline metals, alkaline earth metals, or transition metals, preferably from platinum, molybdenum, iridium, tantalum, bismuth, tungsten, vanadium, cobalt, hafnium, niobium, chromium, manganese, rhenium, iron, gold, silver, copper, ruthenium, rhodium, palladium, osmium, lanthanum and lanthanides, as well as combinations thereof.

In certain exemplary embodiments, the sol/gel forming components may be selected from metal oxides, metal carbides, metal nitrides, metaloxynitrides, metalcarbonitrides, metaloxycarbides, metaloxynitrides, and metaloxycarbonitrides of the above mentioned metals, or any combinations thereof. These compounds, for example in the form of colloidal materials, can e.g. be reacted with oxygen containing compounds, e.g. alkoxides to form a sol/gel.

Sols for metallizing the templates may be derived from at least one sol/gel forming component selected from alkoxides, metal alkoxides, colloidal particles, e.g. metal oxides and the like. Metal alkoxides useful as sol/gel forming components are well-known chemical compounds that are used in a variety of applications. They may have the general formula M(OR)ₓ wherein M is any metal from a metal alkoxide which e.g. will hydrolyze and polymerize to a solid layer in the presence of water. R is an alkyl radical of 1 to 30 carbon atoms, which may be straight chained or branched, and x has a value equivalent to the metal ion valence. In certain exemplary embodiments of the present invention metal alkoxides, such as Si(OR)₄, Ti(OR)₄, Al(OR)₃, Zr(OR)₃ and Sn(OR)₄ may be selected to metallize the templates. For example, R can be a methyl, ethyl, propyl or butyl radical. Further examples of suitable metal alkoxides include Ti(isopropoxy)₄, Al(isopropoxy)₃, Al(sec-butoxy)₃, Zr(n-butoxy)₄ and Zr(n-propoxy)₄.

Further examples can include sols made from silicon alkoxides, such as tetraalkoxysilanes, wherein the alkoxy may be branched or straight chained and may contain 1 to 25 carbon atoms, e.g. tetramethoxysilane (TMOS), tetraethoxysilane (TEOS) or tetra-n-propoxysilane, as well as oligomeric forms thereof. Further examples may include alkylalkoxysilanes, wherein alkoxy is defined as above and alkyl may be a substituted or unsubstituted, branched or straight chain alkyl having 1 to 25 carbon atoms, e.g. methyltrimethoxysilane (MTMOS), methyltriethoxysilane, ethyltriethoxysilane, ethyltrimethoxysilane, methyltripropoxysilane, methyltributoxysilane, propyltrimethoxysilane, propyltriethoxysilane, isobutyltriethoxysilane, isobutyltrimethoxy silane, octyltriethoxysilane, octyltrimethoxysilane, commercially available from Degussa AG, Germany, methacryloxydecyltrimethoxysilane (MDTMS); aryltrialkoxysilanes like phenyltrimethoxysilane (PTMOS), phenyltriethoxysilane, commercially available from Degussa AG, Germany; phenyltripropoxysilane, and phenyltributoxysilane, phenyl-tri-(3-glycidyloxy)-silane-oxide (TGPSO), 3-aminopropyltrimethoxysilane, 3-aminopropyl-triethoxysilane, 2-aminoethyl-3-aminopropyltrimethoxysilane, triaminofunctional propyltrimethoxysilane (Dynasylan® TRIAMO, available from Degussa AG, Germany), N-(n-butyl)-3-aminopropyltrimethoxysilane, 3-aminopropylmethyl-diethoxysilane, 3-glycidyloxypropyltrimethoxysilane, 3-glycidyloxypropyltriethoxy-silane, vinyltrimethoxysilane, vinyltriethoxysilane, 3-mercaptopropyltrimethoxy-silane, Bisphenol-A-glycidylsilanes; (meth)acrylsilanes, phenylsilanes, oligomeric or polymeric silanes, epoxysilanes; fluoroalkylsilanes like fluoroalkyltrimethoxysilanes, fluoroalkyltriethoxysilanes with a partially or fully fluorinated, straight chain or branched fluoroalkyl residue of 1 to 20 carbon atoms, e.g. tridecafluoro-1,1,2,2 tetrahydrooctyltriethoxysilane and modified reactive fluoroalkylsiloxanes available from Degussa AG under the trademarks Dynasylan® F8800 and F8815; as well as any mixtures of the foregoing.

Some of these colloidal sols may be acidic in the sol form and, therefore, when used in conjunction with this invention during hydrolysis, additional acid need not be added to the hydrolysis medium. These colloidal sols may also be prepared by a variety of conventional methods. For example, titania sols having a particle size in the range of 5 to 150 nm can be prepared by the acidic hydrolysis of titanium tetrachloride, by peptizing hydrous TiO₂ with tartaric acid and, by peptizing ammonia washed Ti(SO₄)₂ with hydrochloric acid. See Weiser, Inorganic Colloidal Chemistry, Vol. 2, p. 281 (1935). For the purposes of this invention and in order to preclude the incorporation of contaminants in the sols, the alkyl orthoesters of the metals may be hydrolyzed in an acid pH range of 1 to 3, e.g. in the presence of a water miscible solvent, wherein the colloid may be present in the dispersion in an amount of 0.1 to 10 weight percent.

When the sol is formed by an exemplary hydrolytic sol/gel-process, the molar ratio of the added water and the sol/gel forming components like alkoxides, oxides, acetates, nitrides or combinations thereof, may be in the range of 0.001 to 100, such as from 0.1 to 80, or from 0.2 to 30.

Non-hydrolytic sols can be similarly made as described above, however essentially in the absence of water. In case the sol is formed by a non-hydrolytic sol/gel-process or by chemically linking the components with a linker, the molar ratio of the halide and the oxygen-containing compound may be in the range of about 0.001 to 100, preferable from about 0.1 to 140, even more preferable from about 0.1 to 100, particularly preferable from about 0.2 to 80.

In non-hydrolytic sol/gel processes, the use of metal alkoxides and carboxylic acids and their derivatives or carboxylic acid functionalized polymer-encapsulated active agents may also be used to metallize the template. Suitable carboxylic acids include, e.g. acetic acid, acetoacetic acid, formic acid, maleic acid, crotonic acid, succinic acid, their anhydrides, esters and the like.

In the case of acid anhydrides, it may be preferable to use these anhydrides in admixture with anhydrous alcohols, wherein the molar ratio of these components determines the amount of residual acetoxy groups at the silicon atom of the alkylsilane employed.

To affect a hydrolysis in hydrolytic sol/gel metallization steps, the addition of solvents may be used. Water-miscible solvents may be used, such as water-miscible alcohols or mixtures of water-miscible alcohols, including alcohols, such as methanol, ethanol, n propanol, isopropanol, n-butanol, isobutanol, t-butanol and lower molecular weight ether alcohols, such as ethylene glycol monomethyl ether. Sometimes it may be favorable to use small amounts of non-water-miscible solvents, such as toluene. These solvents can also be used for coating the templates in a metal layer deposition step as described above.

In certain exemplary embodiments, pore sizes and porosity in the metal layer may be controlled by using sol/gel forming metal-based components and a crosslinker. Crosslinkers may include, for example, isocyanates, silanes, diols, di-carboxylic acids, (meth)acrylates, for example, such as 2-hydroxyethyl methacrylate, propyltrimethoxysilane, 3-(trimethylsilyl)propyl methacrylate, isophorone diisocyanate, polyols, glycerine and the like. Furthermore, biocompatible crosslinkers, such as glycerine, diethylene triamino isocyanate and 1,6-diisocyanato hexane or any other suitable cross-linking agent or any mixture thereof may be used.

In certain exemplary embodiments, both electroplating or electroless wet-chemical metal-deposition may be combined with sol-based metal-containing solutions to obtain a porous surface of the material.

Depending on the particular implant and its desired shape, one of the conventional methods available for depositing the metallic layer, or a combination of several methods may be used, if desired. In certain exemplary embodiments, a wet-chemical method including providing the templates in a metal-containing solution may be preferred. Depending on the desired implant and its properties, a suitable metal-source may be selected. For example, producing a magnesium layer on the template typically requires the use of magnesium based salts in solutions or sols or any mixture thereof, in a plating process, or magnesium-based targets or precursors in PVD or CVD methods. Accordingly, to produce a copper coating it is required to use a copper based salt or sol. Selection of the metal source is not limited to the aforesaid metal entities and can be applied to any other metal source that is available in a suitable form, such as a salt, metal, compound, or sol.

In further exemplary embodiments, it may be desirable to mix different metal sources obtaining a mixed metal coating or an alloy layer. In alternative exemplary embodiments, it may be preferable to use different metal species for depositing the metal layer on the templates at once or to produce different layers using the same or different metal sources or any mixture thereof in at least two or even multiple steps.

During deposition of the metal layer, a coating of templates may be carried out conventionally, for example by spray coating, simple dipping of the template into the metal-containing solutions, introducing them into the liquid or a galvanic cell. At time, it may be needed to agitate the template containing metal-based solution. Any known agitating method may be applicable, e.g., using stirrers, ultrasound or the like. In specific exemplary embodiments it may also be possible to spray the template with a metal-containing solution, e.g. by using air nozzles, ultrasound nozzles, atomizers and the like. Alternatively, a template material or part of a template may be first metallized and subsequently formed by conventional molding techniques to the desired template or metallized template.

In certain exemplary embodiments it may be required to dry the metallized templates after deposition of the metal layer. Drying may be carried out by any suitable conventional method, such as heating the material, or drying in a hot air stream.

In certain exemplary embodiments, it may also be desirable to improve the adhesion of the metal layer to the template material, or to improve the integrity of the metal layer, for example by curing. This may be done, for example, thermally by heating, e.g. at a temperature from about 20° to 900° C., such as from about 30° C. to 300° C.

Depending of the materials used the heating may be optionally conducted under an inert gas atmosphere, e.g. to avoid thermal oxidative degradation. Optionally, if oxidation is intended, oxidative conditions can be used to at least partially oxidize the metal layer. In certain exemplary embodiments, the metal layer or the final implant may be stabilized for example by sintering, for example in the temperature range from about 100° C. to 3500° C., such as from about 200° C. to 1000° C., optionally in inert, reactive or different gas atmospheres.

After metallizing, one or a plurality of openings may be introduced into the metallic layer by conventional methods, such as laser cutting, drilling or the like, to provide an access to the template for its removal or for eluting active ingredients from the lumen inside the metallic layer.

The thickness of the metallic layer deposited o the template will depend on the material used, the shape and/or the purpose of the implant. For example, for larger volume reservoirs or large sized implants, the metallic layer may be deposited in a greater thickness than for micro sized implants, such as e.g. stents. As an example, for a stent having a strut thickness of about 100 to 160 μm, an appropriate thickness of the metallic layer may be at about 10 to 20 μm.

In certain exemplary embodiments, for stents, the metallic layer may be made from a cobalt-chromium alloy, a magnesium based alloy, nitinol, or a nickel-titanium alloy. Further preferred metal materials are selected from steel alloys, tantalum alloys or titanium alloys.

### Additives

In certain exemplary embodiments additives may be used for facilitating the metallization process, as explained above. Such additives can be used e.g. to improve wetting of the template, or the chemical or physical adhesion of the metal, etc. Exemplary additives, further to the above mentioned, may include surfactants or emulsifiers like anionic, cationic, zwitter-ionic or non-ionic surfactants and any combinations thereof.

Further additives for wetting, dispersing, or electrostatic stabilizers, rheology or thixotropy modifiers, can include e.g. the various additives sold under the trademarks Byk®, Disperbyk® or Nanobyk® by Byk-Chemie GmbH, Germany, or equivalent compositions from other manufacturers. Other additives may include catalytically active compounds conventionally used in electroplating or electroless plating, as described above, such as cyanides, tin-palladium, palladium, platinum, sensitizers like Sn or Sn ions, and the like.

Other additives to enhance the metallization may be used to chemically modify the templates. Modification my be carried out with suitable linker groups or coatings which are capable to react with the metal layer components. For example, templates may be modified with organosilane compounds or organo-functional silanes.

Exemplary Removal of Template

Removal of the template can result in the formation of at least one hollow space or lumen within the metallic implant. The template may be partially or completely removed after metallization. For removing the template, at least one opening in the metallic layer should be provided in case the template is fully covered by the metallic layer. For porous metallic layers, it may not be necessary to provide an opening.

The template may be removed e.g. by dissolving it in appropriate solvents, particularly if the template material is dissolvable, e.g. an organic compound or polymer, a salt or the like. Suitable solvents may include, for example, (hot) water, diluted or concentrated inorganic or organic acids, bases and the like. Suitable inorganic acids include, for example, hydrochloric acid, sulphuric acid, phosphoric acid, nitric acid as well as diluted hydrofluoric acid. Suitable bases include for example sodium hydroxide, ammonia, carbonate as well as organic amines. Suitable organic acids include, for example, formic acid, acetic acid, trichloromethane acid, trifluoromethane acid, citric acid, tartaric acid, oxalic acid and mixtures thereof. Suitable solvents may comprise, for example, methanol, ethanol, n-propanol, isopropanol, butoxydiglycol, butoxyethanol, butoxyisopropanol, butoxypropanol, n-butyl alcohol, t-butyl alcohol, butylene glycol, butyl octanol, diethylene glycol, dimethoxydiglycol, dimethyl ether, dipropylene glycol, ethoxydiglycol, ethoxyethanol, ethyl hexane diol, glycol, hexane diol, 1,2,6-hexane triol, hexyl alcohol, hexylene glycol, isobutoxy propanol, isopentyl diol, 3-methoxybutanol, methoxydiglycol, methoxyethanol, methoxyisopropanol, methoxymethylbutanol, methoxy PEG-10, methylal, methyl hexyl ether, methyl propane diol, neopentyl glycol, PEG-4, PEG-6, PEG-7, PEG-8, PEG-9, PEG-6-methyl ether, pentylene glycol, PPG-7, PPG-2-buteth-3, PPG-2 butyl ether, PPG-3 butyl ether, PPG-2 methyl ether, PPG-3 methyl ether, PPG-2 propyl ether, propanediol, propylene glycol, propylene glycol butyl ether, propylene glycol propyl ether, tetrahydrofurane, trimethyl hexanol, phenol, benzene, toluene, xylene; as well as water, if necessary in mixture with dispersants, surfactants or other additives and mixtures of the above-named substances.

Another exemplary embodiment may comprise a thermolytic degradation of the template material at elevated temperatures, for example in the range from about 100° C. to 1500° C., such as about 300° C. to 800° C.

Appropriate heating ramps and duration time of the thermal treatment to at least partially remove the template may be selected as desired. For example, the heating may be slow, e.g., the heating ramp may be below about 10 K/min, such as below about 3 K/min or even below about 1 K/min. The thermal treatment may be done in inert gas atmosphere to avoid oxidation of the metal, or in an oxidizing atmosphere like oxygen, carbon monoxide, carbon dioxide, nitrogen oxide. Suitable inert gases can include, e.g., nitrogen, SF₆, or noble gases like argon, or any mixtures thereof. Furthermore, the inert atmosphere may be blended with reactive gases, e.g. hydrogen, ammoniac, C1-C6 saturated aliphatic hydrocarbons like methane, ethane, propane and butene, mixtures thereof or other oxidizing gases. In certain exemplary embodiments, the atmosphere may be substantially free of oxygen. The oxygen content may be below 10 ppm, such as below 1 ppm.

For example, with carbon-based templates, such as polymers, a thermolytic degradation under oxidative atmospheres may be preferred.

In a further exemplary embodiment, the removal of the template may occur in-vivo within the living body after implantation. In these embodiments the template may be selected from bio-corrodible metals or metal oxides or biodegradable polymers as described above.

Exemplary Delivery and Release of Active Ingredients

In an exemplary embodiment, the template itself may comprise at least one active ingredient, such as, for example, a biologically active, therapeutically active, diagnostic or absorptive agent. The active ingredient may be incorporated into or coated onto the template before metallization. Typically, in such embodiments the template may consist of a material which is biodegradable in-vivo, so that release of the active ingredient may occur before or simultaneously with the removal or degradation of the template in-vivo. Alternatively, the biodegradable template may be impregnated or soaked with active ingredients after metallization. For example, this may be done by dipping the template containing implant into a solution of an active ingredient, so that the template may be impregnated with active ingredient through openings or pores in the metallic layer.

In a further exemplary embodiment, the hollow space or reservoirs within the implant device may be filled with an active ingredient after removal of the template. In certain exemplary embodiments, an implant may comprise more than one discrete reservoir which may be filled with different active ingredients separately. Any combinations of these concepts of introducing active ingredients into the implants may be selected as desired.

In addition, the active ingredient may be configured to be released from the implant reservoir in-vivo. For example, release controlling coatings on the implant or a controlled release matrix in the reservoir may be used as desired.

The active ingredients suitable for being incorporated into the implant, or for being coated on at least a part of the implant according to the exemplary embodiment of the present invention may include therapeutically active agents which are capable of providing direct or indirect therapeutic, physiologic and/or pharmacologic effect in a human or animal organism. In an alternative embodiment, the active agent may also be a compound for agricultural purposes, for example a fertilizer, pesticide, microbicide, herbicide, algicide and the like.

The therapeutically active agent may be a drug, pro-drug or even a targeting group or a drug comprising a targeting group.

The active ingredients may be in crystalline, polymorphous or amorphous form or any combination thereof in order to be used in the present invention. Suitable therapeutically active agents may be selected from the group of enzyme inhibitors, hormones, cytokines, growth factors, receptor ligands, antibodies, antigens, ion binding agents, such as crown ethers and chelating compounds, substantial complementary nucleic acids, nucleic acid binding proteins including transcriptions factors, toxins etc. Examples of such active agents are, for example, cytokines, such as erythropoietine (EPO), thrombopoietine (TPO), interleukines (including IL-1 to IL-17), insulin, insulin-like growth factors (including IGF-1 and IGF-2), epidermal growth factor (EGF), transforming growth factors (including TGF-alpha and TGF-beta), human growth hormone, transferrine, low density lipoproteins, high density lipoproteins, leptine, VEGF, PDGF, ciliary neurotrophic factor, prolactine, adrenocorticotropic hormone (ACTH), calcitonin, human chorionic gonadotropin, cortisol, estradiol, follicle stimulating hormone (FSH), thyroid-stimulating hormone (TSH), leutinizing hormone (LH), progesterone, testosterone, toxins including ricine, and further active agents, such as those included in Physician's Desk Reference, 58th Edition, Medical Economics Data Production Company, Montvale, N.J., 2004 and the Merck Index, 13th Edition (particularly pages Ther-1 to Ther-29).

In an exemplary embodiment, the therapeutically active agent can be selected from the group of drugs for the therapy of oncological diseases and cellular or tissue alterations. Suitable therapeutic agents are, e.g., antineoplastic agents, including alkylating agents, such as alkyl sulfonates, e.g., busulfan, improsulfan, piposulfane, aziridines, such as benzodepa, carboquone, meturedepa, uredepa; ethyleneimine and methylmelamines, such as altretamine, triethylene melamine, triethylene phosphoramide, triethylene thiophosphoramide, trimethylolmelamine; so-called nitrogen mustards, such as chlorambucil, chlomaphazine, cyclophosphamide, estramustine, ifosfamide, mechlorethamine, mechlorethaminoxide hydrochloride, melphalan, novembichin, phenesterine, prednimustine, trofosfamide, uracil mustard; nitroso urea-compounds, such as carmustine, chlorozotocin, fotenmustine, lomustine, nimustine, ranimustine; dacarbazine, mannomustine, mitobranitol, mitolactol; pipobroman; doxorubicin and cis-platinum and its derivatives, etc., combinations and/or derivatives of any of the foregoing.

In a further exemplary embodiment, the therapeutically active agent may be selected from the group of anti-viral and anti-bacterial agents, such as aclacinomycin, actinomycin, anthramycin, azaserine, bleomycin, cuctinomycin, carubicin, carzinophilin, chromomycines, ductinomycin, daunorubicin, 6-diazo-5-oxn-1-norieucin, doxorubicin, epirubicin, mitomycins, mycophenolsäure, mogalumycin, olivomycin, peplomycin, plicamycin, porfiromycin, puromycin, streptonigrin, streptozocin, tubercidin, ubenimex, zinostatin, zorubicin, aminoglycosides or polyenes or macrolid-antibiotics, etc., combinations and/or derivatives of any of the foregoing.

In a further exemplary embodiment, the therapeutically active agent may include a radio-sensitizer drug.

In a further exemplary embodiment, the therapeutically active agent may include a steroidal or non-steroidal anti-inflammatory drug.

In a further exemplary embodiment, the therapeutically active agent can be selected from agents referring to angiogenesis, such as e.g. endostatin, angiostatin, interferones, platelet factor 4 (PF4), thrombospondin, transforming growth factor beta, tissue inhibitors of the metalloproteinases-1, -2 and -3 (TIMP-1, -2 and -3), TNP-470, marimastat, neovastat, BMS-275291, COL-3, AG3340, thalidomide, squalamine, combrestastatin, SU5416, SU6668, IFN-[alpha], EMD121974, CAI, IL-12 and IM862 etc., combinations and/or derivatives of any of the foregoing.

In a further exemplary embodiment, the therapeutically-active agent may be selected from the group of nucleic acids, whereas the term nucleic acids can also comprise oliogonucleotides wherein at least two nucleotides are covalently linked to each other, for example in order to provide gene therapeutic or antisense effects. Nucleic acids preferably comprise phosphodiester bonds, which also comprise those which are analogues having different backbones. Analogues may also contain backbones such as, for example, phosphoramide (see publications Beaucage et al., Tetrahedron 49(10):1925 (1993) and the references cited therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)); phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphosphoroamidit-compounds (see Eckstein, Oligonucleotides and Analogues: A Practical Approach, Oxford University Press), and peptide-nucleic acid-backbones and their compounds (see Egholm, J. Am. Chem. Soc. 114:1895 (1992); Meier et al., Chem. Int. Ed. Engl: 31:1008 (1992); Nielsen, Nature, 365:566 (1993); Carlsson et al., Nature 380:207 (1996), whereas these references are incorporated herein by reference. Further analogues are those having ionic backbones, see Denpcy et al., Proc. Natl. Acad. Sci. USA 92:6097 (1995), or non-ionic backbones, see U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996), and non-ribose-backbones, including those which are described in U.S. Pat. Nos. 5,235,033 and 5,034,506, and in chapters 6 and 7 of ASC Symposium Series 580, “Carbohydrate Modifications in Antisense Research”, Ed. Y. S. Sanghui and P. Dan Cook. The nucleic acids having one or more carbocylic sugars are also suitable as nucleic acids for use in the present invention, see Jenkins et al., Chemical Society Review (1995), pages 169 to 176 as well as others which are described in Rawls, C & E News, 2 Jun. 1997, page 36, herewith incorporated by reference. Besides the selection of the nucleic acids and nucleic acid analogues known in the prior art, also a mixture of naturally occurring nucleic acids and nucleic acid analogues or mixtures of nucleic acid analogues may be used.

In a further exemplary embodiment, the therapeutically active agent can be selected from the group of metal ion complexes, as described in International Applications PCT/US95/16377, PCT/US95/16377, PCT/US96/19900, PCT/US96/15527, wherein such agents reduce or inactivate the bioactivity of their target molecules, preferably proteins such as enzymes.

Therapeutically active agents may also include anti-migratory, anti-proliferative or immune-supressive, anti-inflammatory or re-endotheliating agents such as, e.g., everolimus, tacrolimus, sirolimus, mycofenolate-mofetil, rapamycin, paclitaxel, actinomycine D, angiopeptin, batimastate, estradiol, VEGF, statines and others, their derivatives and analogues.

Active agents or combinations of active agents may further be selected from heparin, synthetic heparin analogs (e.g., fondaparinux), hirudin, antithrombin III, drotrecogin alpha; fibrinolytics, such as alteplase, plasmin, lysokinases, factor XIIa, prourokinase, urokinase, anistreplase, streptokinase; platelet aggregation inhibitors, such as acetylsalicylic acid [aspirin], ticlopidine, clopidogrel, abciximab, dextrans; corticosteroids, such as alclometasone, amcinonide, augmented betamethasone, beclomethasone, betamethasone, budesonide, cortisone, clobetasol, clocortolone, desonide, desoximetasone, dexamethasone, fluocinolone, fluocinonide, flurandrenolide, flunisolide, fluticasone, halcinonide, halobetasol, hydrocortisone, methylprednisolone, mometasone, prednicarbate, prednisone, prednisolone, triamcinolone; so-called non-steroidal anti-inflammatory drugs (NSAIDs), such as diclofenac, diflunisal, etodolac, fenoprofen, flurbiprofen, ibuprofen, indomethacin, ketoprofen, ketorolac, meclofenamate, mefenamic acid, meloxicam, nabumetone, naproxen, oxaprozin, piroxicam, salsalate, sulindac, tolmetin, celecoxib, rofecoxib; cytostatics, such as alkaloides and podophyllum toxins, such as vinblastine, vincristine; alkylating agents, such as nitrosoureas, nitrogen lost analogs; cytotoxic antibiotics, such as daunorubicin, doxorubicin and other anthracyclines and related substances, bleomycin, mitomycin; antimetabolites, such as folic acid analogs, purine analogs or pyrimidine analogs; paclitaxel, docetaxel, sirolimus; platinum compounds, such as carboplatin, cisplatin or oxaliplatin; amsacrin, irinotecan, imatinib, topotecan, interferon-alpha 2a, interferon-alpha 2b, hydroxycarbamide, miltefosine, pentostatin, porfimer, aldesleukin, bexaroten, tretinoin; antiandrogens and antiestrogens; antiarrythmics in particular class I antiarrhythmic, such as antiarrhythmics of the quinidine type, quinidine, dysopyramide, ajmaline, prajmalium bitartrate, detajmium bitartrate; antiarrhythmics of the lidocaine type, e.g., lidocaine, mexiletin, phenyloin, tocainid; class Ic antiarrhythmics, e.g., propafenon, flecainid(acetate); class II antiarrhythmics beta-receptor blockers, such as metoprolol, esmolol, propranolol, metoprolol, atenolol, oxprenolol; class III antiarrhythmics, such as amiodarone, sotalol; class IV antiarrhythmics, such as diltiazem, verapamil, gallopamil; other antiarrhythmics, such as adenosine, orciprenaline, ipratropium bromide; agents for stimulating angiogenesis in the myocardium, such as vascular endothelial growth factor (VEGF), basic fibroblast growth factor (bFGF), non-viral DNA, viral DNA, endothelial growth factors: FGF-1, FGF-2, VEGF, TGF; antibiotics, monoclonal antibodies, anticalins; stem cells, endothelial progenitor cells (EPC); digitalis glycosides, such as acetyl digoxin/metildigoxin, digitoxin, digoxin; cardiac glycosides, such as ouabain, proscillaridin; antihypertensives, such as CNS active antiadrenergic substances, e.g., methyldopa, imidazoline receptor agonists; calcium channel blockers of the dihydropyridine type, such as nifedipine, nitrendipine; ACE inhibitors: quinaprilate, cilazapril, moexipril, trandolapril, spirapril, imidapril, trandolapril; angiotensin II antagonists: candesartancilexetil, valsartan, telmisartan, olmesartanmedoxomil, eprosartan; peripherally active alpha-receptor blockers, such as prazosin, urapidil, doxazosin, bunazosin, terazosin, indoramin; vasodilatators, such as dihydralazine, diisopropylamine dichloracetate, minoxidil, nitroprusside sodium; other antihypertensives, such as indapamide, co-dergocrine mesylate, dihydroergotoxin methanessulfonate, cicletanin, bosentan, fludrocortisone; phosphodiesterase inhibitors, such as milrinon, enoximon and antihypotensives, such as in particular adrenergic and dopaminergic substances, such as dobutamine, epinephrine, etilefrine, norfenefrine, norepinephrine, oxilofrine, dopamine, midodrine, pholedrine, ameziniummetil; and partial adrenoceptor agonists, such as dihydroergotamine; fibronectin, polylysine, ethylene vinyl acetate, inflammatory cytokines, such as: TGF, PDGF, VEGF, bFGF, TNF, NGF, GM-CSF, IGF-a, IL-1, IL 8, IL-6, growth hormone; as well as adhesive substances, such as cyanoacrylates, beryllium, silica; and growth factors, such as erythropoetin, hormones, such as corticotropins, gonadotropins, somatropins, thyrotrophins, desmopressin, terlipressin, pxytocin, cetrorelix, corticorelin, leuprorelin, triptorelin, gonadorelin, ganirelix, buserelin, nafarelin, goserelin, as well as regulatory peptides, such as somatostatin, octreotid; bone and cartilage stimulating peptides, bone morphogenetic proteins (BMPs), in particulary recombinant BMPs, such as recombinant human BMP-2 (rhBMP-2), bisphosphonate (e.g., risedronate, pamidronate, ibandronate, zoledronic acid, clodronsaure, etidronsaure, alendronic acid, tiludronic acid), fluorides, such as disodium fluorophosphate, sodium fluoride; calcitonin, dihydrotachystyrol; growth factors and cytokines, such as epidermal growth factor (EGF), platelet-derived growth factor (PDGF), fibroblast growth factors (FGFs), transforming growth factors-b (TGFs-b), transforming growth factor-a (TGF-a), erythropoietin (EPO), insulin-like growth factor-I (IGF-I), insulin-like growth factor-II (IGF-II), interleukin-1 (IL-1), interleukin-2 (IL-2), interleukin-6 (IL-6), interleukin-8 (IL-8), tumor necrosis factor-a (TNF-a), tumor necrosis factor-b (TNF-b), interferon-g (INF-g), colony stimulating factors (CSFs); monocyte chemotactic protein, fibroblast stimulating factor 1, histamine, fibrin or fibrinogen, endothelin-1, angiotensin II, collagens, bromocriptine, methysergide, methotrexate, carbon tetrachloride, thioacetamide and ethanol; as well as silver (ions), titanium dioxide, antibiotics and anti-infective drugs, such as in particular β-lactam antibiotics, e.g., β-lactamase-sensitive penicillins, such as benzyl penicillins (penicillin G), phenoxymethylpenicillin (penicillin V); β-lactamase-resistant penicillins, such as aminopenicillins, e.g., amoxicillin, ampicillin, bacampicillin; acylaminopenicillins, such as mezlocillin, piperacillin; carboxypenicillins, cephalosporins, such as cefazoline, cefuroxim, cefoxitin, cefotiam, cefaclor, cefadroxil, cefalexin, loracarbef, cefixim, cefuroximaxetil, ceftibuten, cefpodoximproxetil, cefpodoximproxetil; aztreonam, ertapenem, meropenem; β-lactamase inhibitors, such as sulbactam, sultamicillintosylate; tetracyclines, such as doxycycline, minocycline, tetracycline, chlorotetracycline, oxytetracycline; aminoglycosides, such as gentamicin, neomycin, streptomycin, tobramycin, amikacin, netilmicin, paromomycin, framycetin, spectinomycin; macrolide antibiotics, such as azithromycin, clarithromycin, erythromycin, roxithromycin, spiramycin, josamycin; lincosamides, such as clindamycin, lincomycin; gyrase inhibitors, such as fluoroquinolones, e.g., ciprofloxacin, ofloxacin, moxifloxacin, norfloxacin, gatifloxacin, enoxacin, fleroxacin, levofloxacin; quinolones, such as pipemidic acid; sulfonamides, trimethoprim, sulfadiazine, sulfalene; glycopeptide antibiotics, such as vancomycin, teicoplanin; polypeptide antibiotics, such as polymyxins, e.g., colistin, polymyxin-b, nitroimidazole derivates, e.g., metronidazole, timidazole; aminoquinolones, such as chloroquin, mefloquin, hydroxychloroquin; biguanids, such as proguanil; quinine alkaloids and diaminopyrimidines, such as pyrimethamine; amphenicols, such as chloramphenicol; rifabutin, dapson, fusidic acid, fosfomycin, nifuratel, telithromycin, fusafingin, fosfomycin, pentamidine diisethionate, rifampicin, taurolidin, atovaquon, linezolid; virus static, such as aciclovir, ganciclovir, famciclovir, foscarnet, inosine-(dimepranol-4-acetamidobenzoate), valganciclovir, valaciclovir, cidofovir, brivudin; antiretroviral active ingredients (nucleoside analog reverse-transcriptase inhibitors and derivatives), such as lamivudine, zalcitabine, didanosine, zidovudin, tenofovir, stavudin, abacavir; non-nucleoside analog reverse-transcriptase inhibitors: amprenavir, indinavir, saquinavir, lopinavir, ritonavir, nelfinavir; amantadine, ribavirine, zanamivir, oseltamivir or lamivudine, as well as any combinations and mixtures thereof.

In an alternative exemplary embodiment of the present invention, the active agents can be encapsulated in polymers, vesicles, liposomes or micelles.

Suitable diagnostically active agents can be e.g. signal generating agents or materials, which may be used as markers. Such signal generating agents are materials which in physical, chemical and/or biological measurement and verification methods lead to detectable signals, for example in image-producing methods. It is not important for the present invention, whether the signal processing is carried out exclusively for diagnostic or therapeutic purposes. Typical imaging methods are for example radiographic methods, which are based on ionizing radiation, for example conventional X-ray methods and X-ray based split image methods, such as computer tomography, neutron transmission tomography, radiofrequency magnetization, such as magnetic resonance tomography, further by radionuclide-based methods, such as scintigraphy, Single Photon Emission Computed Tomography (SPECT), Positron Emission Computed Tomography (PET), ultrasound-based methods or fluoroscopic methods or luminescence or fluorescence based methods, such as Intravasal Fluorescence Spectroscopy, Raman spectroscopy, Fluorescence Emission Spectroscopy, Electrical Impedance Spectroscopy, colorimetry, optical coherence tomography, etc, further Electron Spin Resonance (ESR), Radio Frequency (RF) and Microwave Laser and similar methods.

Signal generating metal-based agents may be incorporated into the metallic layer of the implant or into a structural part of the implant to improve visibility of the implant in the body. For example, semiconducting or magnetic materials, or materials visible, e.g., by x-ray may be incorporated into the metallic layer in exemplary embodiments to mark the implant for better visibility and localization in the body after implantation.

Signal generating agents can be metal-based from the group of metals, metal oxides, metal carbides, metal nitrides, metal oxynitrides, metal carbonitrides, metal oxycarbides, metal oxynitrides, metal oxycarbonitrides, metal hydrides, metal alkoxides, metal halides, inorganic or organic metal salts, metal polymers, metallocenes, and other organometallic compounds.

Exemplary signal generating agents can be especially nanomorphous nanoparticles from metals, metal oxides or semiconductors as defined above, or mixtures thereof.

Further, signal producing materials can be selected from salts or metal ions, which preferably have paramagnetic properties, for example lead (II), bismuth (II), bismuth (III), chromium (III), manganese (II), manganese (III), iron (II), iron (III), cobalt (II), nickel (II), copper (II), praseodymium (III), neodymium (III), samarium (III), or ytterbium (III), holmium (III) or erbium (III) and the like. Based on especially pronounced magnetic moments, especially gadolinium (III), terbium (III), dysprosium (III), holmium (III) and erbium (III) are mostly preferred. Further one can select from radioisotopes. Examples of a few applicable radioisotopes include H 3, Be 10, O 15, Ca 49, Fe 60, In 111, Pb 210, Ra 220, Ra 224 and the like. Typically such ions are present as chelates or complexes, wherein for example as chelating agents or ligands for lanthanides and paramagnetic ions compounds, such as diethylenetriamine pentaacetic acid (“DTPA”), ethylenediamine tetra acetic acid (“EDTA”), or tetraazacyclododecane-N,N′,N″,N′″-tetra acetic acid (“DOTA”) are used. Other typical organic complexing agents are for example published in Alexander, Chem. Rev. 95:273-342 (1995) and Jackels, Pharm. Med. Imag, Section III, Chap. 20, p645 (1990). Other usable chelating agents may be found in U.S. Pat. Nos. 5,155,215; 5,087,440; 5,219,553; 5,188,816; 4,885,363; 5,358,704; 5,262,532, and Meyer et al., Invest. Radiol. 25: S53 (1990), and also U.S. Pat. Nos. 5,188,816, 5,358,704, 4,885,363, and 5,219,553. In addition, salts and chelates from the lanthanide group with the atomic numbers 57-83 or the transition metals with the atomic numbers 21-29, or 42 or 44 may be incorporated into the implants of the exemplary embodiments of the present invention.

Additionally suitable can be paramagnetic perfluoroalkyl containing compounds which for example are described in German laid-open patents DE 196 03 033, DE 197 29 013 and in WO 97/26017, further diamagnetic perfluoroalkyl containing substances of the general formula:

R<PF>-L<II>-G<III>,

whereas R<PF> represents a perfluoroalkyl group with 4 to 30 carbon atoms, L<II> stands for a linker and G<III> for a hydrophilic group. The linker L is a direct bond, an —SO2-group or a straight or branched carbon chain with up to 20 carbon atoms which can be substituted with one or more —OH, —COO<—>, —SO3-groups and/or if necessary one or more —O—, —S—, —CO—, —CONH—, —NHCO—, —CONR—, —NRCO—, —SO2-, —PO4-, —NH—, —NR-groups, an aryl ring or contain a piperazine, wherein R stands for a C1 to C20 alkyl group, which again can contain and/or have one or a plurality of O atoms and/or be substituted with —COO<—> or SO3-groups.

The hydrophilic group G<III> can be selected from a mono or disaccharide, one or a plurality of —COO<—> or —SO3<—>-groups, a dicarboxylic acid, an isophthalic acid, a picolinic acid, a benzenesulfonic acid, a tetrahydropyranedicarboxylic acid, a 2,6-pyridinedicarboxylic acid, a quaternary ammonium ion, an aminopolycarboxcylic acid, an aminodipolyethyleneglycol sulfonic acid, an aminopolyethyleneglycol group, an SO2-(CH2)2-OH-group, a polyhydroxyalkyl chain with at least two hydroxyl groups or one or a plurality of polyethylene glycol chains having at least two glycol units, wherein the polyethylene glycol chains are terminated by an —OH or —OCH3-group, or similar linkages.

In exemplary embodiments paramagnetic metals in the form of metal complexes with phthalocyanines may be used to functionalize the implant, e.g., as described in Phthalocyanine Properties and Applications, Vol. 14, C. C. Leznoff and A. B. P. Lever, VCH Ed. Examples are octa(1,4,7,10-tetraoxaundecyl)Gd-phthalocyanine, octa(1,4,7,10-tetraoxaundecyl)Gd-phthalocyanine, octa(1,4,7,10-tetraoxaundecyl)Mn-phthalocyanine, octa(1,4,7,10-tetraoxaundecyl)Mn-phthalocyanine, as described in U.S. Patent Publication No. 2004/214810.

super-paramagnetic, ferromagnetic or ferrimagnetic signal generating agents may also be used. For example among magnetic metals, alloys are preferred, among ferrites, such as gamma iron oxide, magnetites or cobalt-, nickel- or manganese-ferrites, corresponding agents are preferably selected, especially particles as described in International Patent Publications WO83/03920, WO83/01738, WO85/02772 and WO89/03675, in U.S. Pat. Nos. 4,452,773 and 4,675,173, in WO88/00060 as well as U.S. Pat. No. 4,770,183, and in International Patent Publications WO90/01295 and WO90/01899.

Further, magnetic, paramagnetic, diamagnetic or super paramagnetic metal oxide crystals having diameters of less than 4000 Angstroms can be preferable as degradable non-organic diagnostic agents. Suitable metal oxides can be selected from iron oxide, cobalt oxides, iridium oxides or the like, which provide suitable signal producing properties and which have especially biocompatible properties or are likely biodegradable. Crystalline agents of this group having diameters smaller than 500 Angstroms may be used. These crystals can be associated covalently or non-covalently with macromolecular species. Further, zeolites containing paramagnets and gadolinium containing nanoparticles can be selected from polyoxometallates, preferably of the lanthanides, (e.g., K9GdW10O36).

For optimizing the image producing properties the average particle size of the magnetic signal producing agents may be limited to about 5 μm at maximum, such as from about 2 nm up to 1 μm, e.g. from about 5 nm to 200 nm. The super paramagnetic signal producing agents can be selected for example from the group of so-called SPIOs (super paramagnetic iron oxides) with a particle size larger than about 50 nm or from the group of the USPIOs (ultra small super paramagnetic iron oxides) with particle sizes smaller than about 50 nm.

Signal generating agents for imparting further functionality to the implants of embodiments of the present invention can further be selected from endohedral fullerenes, as described, for example, in U.S. Pat. No. 5,688,486 or International Patent Publication WO 93/15768, or from fullerene derivatives and their metal complexes, such as fullerene species, which comprise carbon clusters having 60, 70, 76, 78, 82, 84, 90, 96 or more carbon atoms. An overview of such species can be obtained from European Patent Application 1331226A2. Metal fullerenes or endohedral carbon-carbon nanoparticles with arbitrary metal-based components can also be selected. Such endohedral fullerenes or endometallo fullerenes may contain for example rare earths, such as cerium, neodymium, samarium, europium, gadolinium, terbium, dysprosium or holmium. The choice of nanomorphous carbon species is not limited to fullerenes, other nanomorphous carbon species, such as nanotubes, onions, etc. may also be applicable.

In another exemplary embodiment fullerene species may be selected from non-endohedral or endohedral forms which contain halogenated, preferably iodated, groups, as described in U.S. Pat. No. 6,660,248.

Generally, mixtures of such signal generating agents of different specifications can also used, depending on the desired properties of the signal generating material properties. The signal producing agents used can have a size of about 0.5 nm to 1,000 nm, preferably about 0.5 nm to 900 nm, especially preferably from about 0.7 to 100 nm, and the may partly replace the metal-based particles. Nanoparticles can be easily modifiable based on their large surface to volume ratios. The nanoparticles can, for example, be modified non-covalently by means of hydrophobic ligands, for example with trioctylphosphine, or be covalently modified. Examples of covalent ligands are thiol fatty acids, amino fatty acids, fatty acid alcohols, fatty acids, fatty acid ester groups or mixtures thereof, for example oleic acid and oleylamine.

In exemplary embodiments of the present invention, the signal producing agents can be encapsulated in micelles or liposomes with the use of amphiphilic components, or may be encapsulated in polymeric shells, for example to be incorporated into the reservoir for co-release with other active ingredients. The micelles/liposomes can have a diameter of about 2 nm to 800 nm, preferably from about 5 to 200 nm, especially preferably from about 10 to 25 nm. The micelles/liposomes may also be added to the template, to be incorporated into the implant.

Signal generating agents may also be selected from non-metal-based signal generating agents, for example from the group of X-ray contrast agents, which can be ionic or non-ionic. Among the ionic contrast agents can be included salts of 3-acetyl amino-2,4-6-triiodobenzoic acid, 3,5-diacetamido-2,4,6-triiodobenzoic acid, 2,4,6-triiodo-3,5-dipropionamido-benzoic acid, 3-acetyl amino-5-((acetyl amino)methyl)-2,4,6-triiodobenzoic acid, 3-acetyl amino-5-(acetyl methyl amino)-2,4,6-triiodobenzoic acid, 5-acetamido-2,4,6-triiodo-N-((methylcarbamoyl)methyl)-isophthalamic acid, 5-(2-methoxyacetamido)-2,4,6-triiodo-N-[2-hydroxy-1-(methylcarbamoyl)-ethoxy 1]-isophthalamic acid, 5-acetamido-2,4,6-triiodo-N-methylisophthalamic acid, 5-acetamido-2,4,6-triiodo-N-(2-hydroxyethyl)-isophthalamic acid 2-[[2,4,6-triiodo-3-[(1-oxobutyl)-amino]phenyl]methyl]-butanoic acid, beta-(3-amino-2,4,6-triiodophenyl)-alpha-ethyl-propanoic acid, 3-ethyl-3-hydroxy-2,4,6-triiodophenyl-propanoic acid, 3-[[(dimethylamino)-methyl]amino]-2,4,6-triiodophenyl-propanoic acid (see Chem. Ber. 93: 2347 (1960)), alpha-ethyl-(2,4,6-triiodo-3-(2-oxo-1-pyrrolidinyl)-phenyl)-propanoic acid, 2-[2-[3-(acetyl amino)-2,4,6-triiodophenoxy]ethoxymethyl]butanoic acid, N-(3-amino-2,4,6-triiodobenzoyl)-N-phenyl-.beta.-aminopropanoic acid, 3-acetyl-[(3-amino-2,4,6-triiodophenyl)amino]-2-methylpropanoic acid, 5-[(3-amino-2,4,6-triiodophenyl)methyl amino]-5-oxypentanoic acid, 4-[ethyl-[2,4,6-triiodo-3-(methyl amino)-phenyl]amino]-4-oxo-butanoic acid, 3,3′-oxy-bis[2,1-ethanediyloxy-(1-oxo-2,1-ethanediyl)imino]bis-2,4,6-triiodobenzoic acid, 4,7,10,13-tetraoxahexadecane-1,16-dioyl-bis(3-carboxy-2,4,6-triiodoanilide), 5,5′-(azelaoyldiimino)-bis[2,4,6-triiodo-3-(acetyl amino)methyl-benzoic acid], 5,5′-(apidoldiimino)bis(2,4,6-triiodo-N-methyl-isophthalamic acid), 5,5′-(sebacoyl-diimino)-bis(2,4,6-triiodo-N-methylisophthalamic acid), 5,5-[N,N-diacetyl-(4,9-dioxy-2,11-dihydroxy-1,12-dodecanediyl)diimino]bis(2,4,6-triiodo-N-methyl-isophthalamic acid), 5,5′5″-(nitrilo-triacetyltriimino)tris(2,4,6-triiodo-N-methyl-isophthalamic acid), 4-hydroxy-3,5-diiodo-alpha-phenylbenzenepropanoic acid, 3,5-diiodo-4-oxo-1(4H)-pyridine acetic acid, 1,4-dihydro-3,5-diiodo-1-methyl-4-oxo-2,6-pyridinedicarboxylic acid, 5-iodo-2-oxo-1 (2H)-pyridine acetic acid, and N-(2-hydroxyethyl)-2,4,6-triiodo-5-[2,4,6-triiodo-3-(N-methylacetamido)-5-(methylcarbomoyl)benzamino]acetamido]-isophthalamic acid, and the like, especially preferred, as well as other ionic X-ray contrast agents suggested in the literature, for example in J. Am. Pharm. Assoc., Sci. Ed. 42:721 (1953), Swiss Patent 480071, JACS 78:3210 (1956), German patent 2229360, U.S. Pat. No. 3,476,802, Arch. Pharm. (Weinheim, Germany) 306: 11 834 (1973), J. Med. Chem. 6: 24 (1963), FR-M-6777, Pharmazie 16: 389 (1961), U.S. Pat. No. 2,705,726, U.S. Pat. No. 2,895,988, Chem. Ber. 93:2347 (1960), SA-A-68/01614, Acta Radiol. 12: 882 (1972), British Patent 870321, Rec. Trav. Chim. 87: 308 (1968), East German Patent 67209, German Patent 2050217, German Patent 2405652, Farm Ed. Sci. 28: 912 (1973), Farm Ed. Sci. 28: 996 (1973), J. Med. Chem. 9: 964 (1966), Arzheim.-Forsch 14: 451 (1964), SE-A-344166, British Patent 1346796, U.S. Pat. No. 2,551,696, U.S. Pat. No. 1,993,039, Ann 494: 284 (1932), J. Pharm. Soc. (Japan) 50: 727 (1930), and U.S. Pat. No. 4,005,188.

Examples of applicable non-ionic X-ray contrast agents in accordance with the present invention are metrizamide as disclosed in German Patent Publication A-2031724, iopamidol as disclosed in BE-A-836355, iohexyl as disclosed in Great Britain Patent Publication A-1548594, iotrolan as disclosed in European Patent Publication A-33426, iodecimol as disclosed in European Patent Publication A-49745, iodixanol as in EP-A-108638, ioglucol as disclosed in U.S. Pat. No. 4,314,055, ioglucomide as described in BE-A-846657, ioglunioe as in German Patent Publication A-2456685, iogulamide as in BE-A-882309, iomeprol as in European Patent Publication A-26281, iopentol as European Patent Publication A-105752, iopromide as in German Patent Publication A-2909439, iosarcol as in DE-A-3407473, iosimide as in German Patent Publication A-3001292, iotasul as in European Patent Publication A-22056, iovarsul as described in European Patent Publication A-83964 or ioxilan as described in International Publication WO87/00757.

Agents based on nanoparticle signal generating agents may be selected to impart functionality to the implant, which after release into tissues and cells are incorporated or are enriched in intermediate cell compartments and/or have an especially long residence time in the organism.

Such particles can include water-insoluble agents, a heavy element, such as iodine or barium, PH-50 as monomer, oligomer or polymer (iodinated aroyloxy ester having the empirical formula C19H23I3N2O6, and the chemical names 6-ethoxy-6-oxohexy-3,5-bis(acetyl amino)-2,4,6-triiodobenzoate), an ester of diatrizoic acid, an iodinated aroyloxy ester, or combinations thereof. Particle sizes which can be incorporated by macrophages may be preferred. A corresponding method for this is disclosed in WO03/039601 and suitable agents are described in the publications U.S. Pat. Nos. 5,322,679, 5,466,440, 5,518,187, 5,580,579, and 5,718,388. Nanoparticles which are marked with signal generating agents or such signal generating agents, such as PH-50, which accumulate in intercellular spaces and can make interstitial as well as extrastitial compartments visible, can be advantageous.

Signal generating agents may also include anionic or cationic lipids, as described in U.S. Pat. No. 6,808,720, for example, anionic lipids, such as phosphatidyl acid, phosphatidyl glycerol and their fatty acid esters, or amides of phosphatidyl ethanolamine, such as anandamide and methanandamide, phosphatidyl serine, phosphatidyl inositol and their fatty acid esters, cardiolipin, phosphatidyl ethylene glycol, acid lysolipids, palmitic acid, stearic acid, arachidonic acid, oleic acid, linoleic acid, linolenic acid, myristic acid, sulfolipids and sulfatides, free fatty acids, both saturated and unsaturated and their negatively charged derivatives, etc. Moreover, halogenated, in particular fluorinated anionic lipids can be preferred in exemplary embodiments. The anionic lipids preferably contain cations from the alkaline earth metals beryllium (Be<+2>), magnesium (Mg<+2>), calcium (Ca<+2>), strontium (Sr<+2>) and barium (Ba<+2>), or amphoteric ions, such as aluminium (Al<+3>), gallium (Ga<+3>), germanium (Ge<+3>), tin (Sn+<4>) or lead (Pb<+2> and Pb<+4>), or transition metals, such as titanium (Ti<+3> and Ti<+4>), vanadium (V<+2> and V<+3>), chromium (Cr<+2> and Cr<+3>), manganese (Mn<+2> and Mn<+3>), iron (Fe<+2> and Fe<+3>), cobalt (Co<+2> and Co<+3>), nickel (Ni<+2> and Ni<+3>), copper (Cu<+2>), zinc (Zn<+2>), zirconium (Zr<+4>), niobium (Nb<+3>), molybdenum (Mo<+2> and Mo<+3>), cadmium (Cd<+2>), indium (In <+3>), tungsten (W<+2> and W<+4>), osmium (Os<+2>, Os<+3> and Os<+4>), iridium (Ir<+2>, Ir<+3> and Ir<+4>), mercury (Hg<+2>) or bismuth (Bi<+3>), and/or rare earths, such as lanthanides, for example lanthanum (La<+3>) and gadolinium (Gd<+3>). Cations can include calcium (Ca<+2>), magnesium (Mg<+2>) and zinc (Zn<+2>) and paramagnetic cations such as manganese (Mn<+2>) or gadolinium (Gd<+3>).

Cationic lipids may include phosphatidyl ethanolamine, phospatidylcholine, Glycero-3-ethylphosphatidylcholine and their fatty acid esters, di- and tri-methylammoniumpropane, di- and tri-ethylammoniumpropane and their fatty acid esters, and also derivatives, such as N-[1-(2,3-dioleoyloxy)propyl]-N,N,N-trimethylammonium chloride (“DOTMA”); furthermore, synthetic cationic lipids based on for example naturally occurring lipids, such as dimethyldioctadecylammonium bromide, sphingolipids, sphingomyelin, lysolipids, glycolipids, such as, for example, gangliosides GM1, sulfatides, glycosphingolipids, cholesterol and cholesterol esters or salts, N-succinyldioleoylphosphattidyl ethanolamine, 1,2-dioleoyl-sn-glycerol, 1,3-dipalmitoyl-2-succinylglycerol, 1,2-dipalmitoyl-sn-3-succinylglycerol, 1-hexadecyl-2-palmitoylglycerophosphatidyl ethanolamine and palmitoyl-homocystein, and fluorinated, derivatized cationic lipids, as disclosed in U.S. Ser. No. 08/391,938. Such lipids are furthermore suitable as components of signal generating liposomes, which especially can have pH-sensitive properties as disclosed in U.S. 2004197392 and incorporated herein explicitly.

Other exemplary signal generating agents can be selected from agents, which are transformed into signal generating agents in organisms by means of in-vitro or in-vivo cells, cells as a component of cell cultures, of in-vitro tissues, or cells as a component of multicellular organisms, such as, for example, fungi, plants or animals, in exemplary embodiments from mammals, such as mice or humans. Such agents can be made available in the form of vectors for the transfection of multicellular organisms, wherein the vectors contain recombinant nucleic acids for the coding of signal generating agents. In certain exemplary embodiments, this may be done with signal generating agents, such as metal binding proteins. It can be possible to choose such vectors from the group of viruses for example from adeno viruses, adeno virus associated viruses, herpes simplex viruses, retroviruses, alpha viruses, pox viruses, arena-viruses, vaccinia viruses, influenza viruses, polio viruses or hybrids of any of the above.

Such signal generating agents may be used in combination with delivery systems, e.g. in order to incorporate nucleic acids, which are suitable for coding for signal generating agents, into the target structure. Virus particles for the transfection of mammalian cells may be used, wherein the virus particle contains one or a plurality of coding sequence/s for one or a plurality of signal generating agents as described above. In these exemplary cases, the particles can be generated from one or a plurality of the following viruses: adeno viruses, adeno virus associated viruses, herpes simplex viruses, retroviruses, alpha viruses, pox viruses, arena-viruses, vaccinia viruses, influenza viruses and polio viruses.

These signal generating agents can be made available from colloidal suspensions or emulsions, which are suitable to transfect cells, preferably mammalian cells, wherein these colloidal suspensions and emulsions contain those nucleic acids which possess one or a plurality of the coding sequence(s) for signal generating agents. Such colloidal suspensions or emulsions can include macromolecular complexes, nano capsules, micro spheres, beads, micelles, oil-in-water- or water-in-oil emulsions, mixed micelles and liposomes or any desired mixture of the above.

In addition, cells, cell cultures, organized cell cultures, tissues, organs of desired species and non-human organisms can be chosen which contain recombinant nucleic acids having coding sequences for signal generating agents. In exemplary embodiments organisms can include mouse, rat, dog, monkey, pig, fruit fly, nematode worms, fish or plants or fungi. Further, cells, cell cultures, organized cell cultures, tissues, organs of desired species and non-human organisms can contain one or a plurality of vectors as described above.

Signal generating agents can also be produced in vivo from proteins and made available as described above. Such agents can be directly or indirectly signal producing, while the cells produce (direct) a signal producing protein through transfection, or produce a protein which induces (indirect) the production of a signal producing protein. These signal generating agents are e.g. detectable in methods such as MRI while the relaxation times T1, T2, or both are altered and lead to signal producing effects which can be processed sufficiently for imaging. Such proteins can include protein complexes, such as metalloprotein complexes. Direct signal producing proteins can include such metalloprotein complexes which are formed in the cells. Indirect signal producing agents can include proteins or nucleic acids, for example, which regulate the homeostasis of iron metabolism, the expression of endogenous genes for the production of signal generating agents, and/or the activity of endogenous proteins with direct signal generating properties, for example Iron Regulatory Protein (IRP), transferrin receptor (for the take-up of Fe), erythroid-5-aminobevulinate synthase (for the utilization of Fe, H-Ferritin and L-Ferritin for the purpose of Fe storage). In exemplary embodiments both types of signal generating agents, that is direct and indirect, may be combined with each other, for example an indirect signal generating agent, which regulates the iron-homeostasis and a direct agent, which represents a metal binding protein.

In certain exemplary embodiments, where metal-binding polypeptides are selected as indirect agents, it can be advantageous if the polypeptide binds to one or a plurality of metals which possess signal generating properties. Metals with unpaired electrons in the Dorf orbitals may be used, such as, for example, Fe, Co, Mn, Ni, Gd etc., whereasn especially Fe is available in high physiological concentrations in organisms. Such agents may form metal-rich aggregates, for example crystalline aggregates, whose diameters are larger than about 10 picometers, preferably larger than about 100 picometers, 1 nm, 10 nm or specially preferably larger than about 100 nm.

Further, metal-binding compounds, which have sub-nanomolar affinities with dissociation constants of less than about 10-15 M, 10-2 M or smaller may be used to impart functionality for the implant. Typical polypeptides or metal-binding proteins are lactoferrin, ferritin, or other dimetallocarboxylate proteins, or so-called metal catcher with siderophoric groups, such as hemoglobin. A possible exemplary method for preparation of such signal generating agents, their selection and the possible direct or indirect agents which are producible in vivo and are suitable as signal generating agents is described in International Publication WO 03/075747.

Another group of signal generating agents can be photophysically signal producing agents which consist of dyestuff-peptide-conjugates. Such dyestuff-peptide-conjugates can provide a wide spectrum of absorption maxima, for example polymethin dyestuffs, such as cyanine-, merocyanine-, oxonol- and squarilium dyestuffs. From the class of the polymethin dyestuffs the cyanine dyestuffs, e.g. the indole structure based indocarbo-, indodicarbo- and indotricarbocyanines, can be suitable. Such dyestuffs can be substituted with suitable linking agents and can be functionalized with other groups as desired, see also DE 19917713.

The signal generating agents can further be functionalized as desired. The functionalization by means of so-called “Targeting” groups is meant to include functional chemical compounds which link the signal generating agent or its specifically available form (encapsulation, micelles, micro spheres, vectors etc.) to a specific functional location, or to a determined cell type, tissue type or other desired target structures. Targeting groups can permit the accumulation of signal-producing agents in or at specific target structures. Therefore the targeting groups can be selected from such substances, which are principally suitable to provide a purposeful enrichment of the signal generating agents in their specifically available form by physical, chemical or biological routes or combinations thereof. Useful targeting groups can therefore include antibodies, cell receptor ligands, hormones, lipids, sugars, dextrane, alcohols, bile acids, fatty acids, amino acids, peptides and nucleic acids, which can be chemically or physically attached to signal-generating agents, in order to link the signal-generating agents into/onto a specifically desired structure. Exemplary targeting groups may include those which enrich signal-generating agents in/on a tissue type or on surfaces of cells. Here may not be necessary for the function, that the signal generating agent be taken up into the cytoplasm of the cells. Peptides can be targeting groups, for example chemotactic peptides that are used to visualize inflammation reactions in tissues by means of signal generating agents; see also WO 97/14443.

Antibodies can be used, including antibody fragments, Fab, Fab2, Single Chain Antibodies (for example Fv), chimerical antibodies, moreover antibody-like substances, for example so-called anticalines, wherein it may not be important whether the antibodies are modified after preparation, recombinants are produced or whether they are human or non-human antibodies. Humanized or human antibodies may be used, such as chimerical immunoglobulines, immunoglobulin chains or fragments (such as Fv, Fab, Fab′, F(ab″)2 or other antigen-binding subsequences of antibodies, which may partly contain sequences of non-human antibodies; humanized antibodies may include human immunoglobulines (receptor or recipient antibody), in which groups of a CDR (Complementary Determining Region) of the receptor are replaced through groups of a CDR of a non-human (spender or donor antibody), wherein the spender species for example, mouse, rabbit or other has appropriate specificity, affinity, and capacity for the binding of target antigens. In a few forms the Fv framework groups of the human immunglobulines are replaced by means of corresponding non-human groups. Humanized antibodies can moreover contain groups which either do not occur in either the CDR or Fv framework sequence of the spender or the recipient. Humanized antibodies essentially comprise substantially at least one or preferably two variable domains, in which all or substantial components of the CDR components of the CDR regions or Fv framework sequences correspond with those of the non-human immunoglobulin, and all or substantial components of the FR regions correspond with a human consensus-sequence. Targeting groups can also include hetero-conjugated antibodies. The functions of the selected antibodies or peptides include cell surface markers or molecules, particularly of cancer cells, wherein here a large number of known surface structures are known, such as HER2, VEGF, CA15-3, CA 549, CA 27.29, CA 19, CA 50, CA242, MCA, CA125, DE-PAN-2, etc.

Moreover, targeting groups may contain the functional binding sites of ligands and which are suitable for binding to any desired cell receptors. Examples of target receptors include receptors of the group of insulin receptors, insulin-like growth factor receptor (e IGF-1 and IGF-2), growth hormone receptor, glucose transporters (particularly GLUT 4 receptor), transferrin receptor (transferrin), Epidermal Growth Factor receptor (EGF), low density lipoprotein receptor, high density lipoprotein receptor, leptin receptor, estrogen receptor; interleukin receptors including IL-1, IL-2, IL-3, IL-4, IL-5, IL-6, IL-7, IL-8, IL-9, IL-11, IL-12, IL-13, IL-15, and IL-17 receptor, VEGF receptor (VEGF), PDGF receptor (PDGF), Transforming Growth Factor receptor (including TGF-[alpha] and TGF-[beta]), EPO receptor (EPO), TPO receptor (TPO), ciliary neurotrophic factor receptor, prolactin receptor, and T-cell receptors.

Additionally, hormone receptors may be used, especially for hormones, such as steroidal hormones or protein- or peptide-based hormones, for example, epinephrines, thyroxines, oxytocine, insulin, thyroid-stimulating hormone, calcitonine, chorionic gonadotropine, corticotropine, follicle stimulating hormone, glucagons, leuteinizing hormone, lipotropine, melanocyte-stimulating hormone, norepinephrines, parathyroid hormone, Thyroid-Stimulating Hormone (TSH), vasopressin's, encephalin, serotonin, estradiol, progesterone, testosterone, cortisone, and glucocorticoide. Receptor ligands include those which are on the cell surface receptors of hormones, lipids, proteins, glycol proteins, signal transducers, growth factors, cytokine, and other bio molecules. Moreover, targeting groups can be selected from carbohydrates with the general formula: Cx(H2O)y, wherein herewith also monosaccharides, disaccharides and oligo- as well as polysaccharides are included, as well as other polymers which consist of sugar molecules which contain glycosidic bonds. Carbohydrates may include those in which all or parts of the carbohydrate components contain glycosylated proteins, including the monomers and oligomers of galactose, mannose, fructose, galactosamine, glucosamine, glucose, sialic acid, and the glycosylated components, which make possible the binding to specific receptors, especially cell surface receptors. Other useful carbohydrates include monomers and polymers of glucose, ribose, lactose, raffinose, fructose and other biologically occurring carbohydrates especially polysaccharides, for example, arabinogalactan, gum Arabica, mannan etc., which are suitable for introducing signal generating agents into cells, see U.S. Pat. No. 5,554,386.

Furthermore, targeting groups can include lipids, fats, fatty oils, waxes, phospholipids, glycolipids, terpenes, fatty acids and glycerides, and triglycerides, or eicosanoides, steroids, sterols, suitable compounds of which can also be hormones, such as prostaglandins, opiates and cholesterol etc. All functional groups can be selected as the targeting group, which possess inhibiting properties, such as for example enzyme inhibitors, preferably those which link signal generating agents into/onto enzymes.

Targeting groups can also include functional compounds which enable internalization or incorporation of signal generating agents in the cells, especially in the cytoplasm or in specific cell compartments or organelles, such as, for example, the cell nucleus. For example, such a targeting group may contains all or parts of HIV-1 tat-proteins, their analogs and derivatized or functionally similar proteins, and in this way allows an especially rapid uptake of substances into the cells. As an example refer to Fawell et al., PNAS USA. 91:664 (1994); Frankel et al., Cell 55:1189, (1988); Savion et al., J. Biol. Chem. 256:1149 (1981); Derossi et al., J. Biol. Chem. 269:10444 (1994); and Baldin et al., EMBO J. 9:1511 (1990).

Targeting groups can further include the so-called Nuclear Localisation Signal (NLS), which include positively charged (basic) domains which bind to specifically targeted structures of cell nuclei. Numerous NLS and their amino acid sequences are known including single basic NLS, such as that of the SV40 (monkey virus) large T Antigen (pro Lys Lys Lys Arg Lys Val), Kalderon (1984), et al., Cell, 39:499-509), the teinoic acid receptor-[beta] nuclear localization signal (ARRRRP); NFKB p50 (EEVQRKRQKL; Ghosh et al., Cell 62:1019 (1990); NFKB p65 (EEKRKRTYE; Nolan et al., Cell 64:961 (1991), as well as others (see for example Boulikas, J. Cell. Biochem. 55(1):32-58 (1994), and double basic NLS's, such as for example xenopus (African clawed toad) proteins, nucleoplasmin (Ala Val Lys Arg Pro Ala Ala Thr Lys Lys Ala Gly Gln Ala Lys Lys Lys Lys Leu Asp), Dingwall, et al., Cell, 30:449-458, 1982 and Dingwall, et al., J. Cell Biol., 107:641-849, 1988. Numerous localization studies have shown that NLSs, which are built into synthetic peptides which normally do not address the cell nucleus or were coupled to reporter proteins, lead to an enrichment of such proteins and peptides in cell nuclei. Exemplary references are made to Dingwall, and Laskey, Ann, Rev. Cell Biol., 2:367-390, 1986; Bonnerot, et al., Proc. Natl. Acad. Sci. USA, 84:6795-6799, 1987; Galileo, et al., Proc. Natl. Acad. Sci. USA, 87:458-462, 1990. Targeting groups for the hepatobiliary system may be selected, as suggested in U.S. Pat. Nos. 5,573,752 and 5,582,814.

In exemplary embodiments according to the present invention, the implant can comprise absorptive agents, e.g., to remove compounds from body fluids. Suitable absorptive agents include chelating agents, such as penicillamine, methylene tetramine dihydrochloride, EDTA, DMSA or deferoxamine mesylate, any other appropriate chemical modification, antibodies, and microbeads or other materials containing cross linked reagents for absorption of drugs, toxins or other agents.

According to this invention, at least one active ingredient, such as a therapeutically active agent, diagnostic active agent or absorptive agent or any mixture thereof may partially or completely be incorporated into at least one of a template, lumen or reservoir of the implant. Incorporation may be carried out by any suitable means, such as impregnating, diffusion techniques, dipping, dip-coating, spray-coating injection or the like. The active ingredient may be provided in an appropriate solvent, optionally using additives. The loading of these agents may be carried out under atmospheric, sub-atmospheric pressure or under vacuum. Alternatively, loading may be carried out under high pressure. Incorporation of the active ingredient may be carried out by applying electrical charge to the implant or exposing at least a portion of the implant to a gaseous material including the gaseous or vapour phase of the solvent in which an agent is dissolved or other gases that have a high degree of solubility in the loading solvent. In exemplary embodiments the active ingredients may be provided using carriers that are incorporated into the lumen of the implant. Carriers can e.g. include any suitable polymer or solvent or solvent system as mentioned herein before.

Examples for carriers can include polymers, such as biocompatible polymers, for example, however not exclusively, collagens, albumin, gelatin, hyaluronic acid, starch, cellulose (methylcellulose, hydroxypropylcellulose, hydroxypropylmethylcellulose, carboxymethylcellulose phthalate; further casein, dextran, polysaccharides, fibrinogen, poly(D,L-lactide), poly(D,L-lactide-coglycolide), poly(glycolide), poly(hydroxybutylate), poly(alkyl carbonate), poly(orthoesters), polyesters, poly(hydroxyvaleric acid), polydioxanone, poly(ethyleneterephthalate), poly(malic acid), poly(tartronic acid), polyanhydride, polyphosphohazene, poly(amino acids), and all their copolymers or any mixtures. In certain exemplary embodiments carriers may be selected from pH-sensitive polymers, such as poly(acrylic acid) and derivatives, for example: homopolymers, such as poly(amino carboxylic acid), poly(acrylic acid), poly(methyl acrylic acid) and their copolymers. This may apply likewise for polysaccharides, such as celluloseacetatephthalate, hydroxypropylmethylcellulosephthalate, hydroxypropylmethylcellulosesuccinate, celluloseacetatetrimellitate and chitosan. In certain exemplary embodiments it can be especially preferred to select carriers from temperature sensitive polymers, such as, for example, poly(N-isopropylacrylamide-co-sodium-acrylate-co-n-N-alkylacrylamide), poly(N-methyl-N-n-propylacrylamide), poly(N-methyl-N-isopropylacrylamide), poly(N—N-propylmethacrylamide), poly(N-isopropylacrylamide), poly(N,N-diethylacrylamide), poly(N-isopropylmethacrylamide), poly(N-cyclopropylacrylamide), poly(N-ethylacrylamide), poly(N-ethylmethylacrylamide), poly(N-methyl-N-ethylacrylamide), poly(N-cyclopropylacrylamide). Other polymers suitable to be used as a carrier with thermogel characteristics may include hydroxypropylcellulose, methylcellulose, hydroxypropylmethylcellulose, ethylhydroxyethylcellulose and pluronics like F-127, L-122, L-92, L-81, L-61.

Other carrier polymers can include functionalized styrene, like amino styrene, functionalized dextrane and polyamino acids. Furthermore polyamino acids, (poly-D-amino acids as well as poly-L-amino acids), for example polylysine, and polymers which contain lysine or other suitable amino acids. Other useful polyamino acids are polyglutamic acids, polyaspartic acid, copolymers of lysine and glutamine or aspartic acid, copolymers of lysine with alanine, tyrosine, phenylalanine, serine, tryptophan and/or proline.

Functional modification can also be implemented by adding therapeutically active agents, diagnostic and/or absorptive agents partially or completely to the surface of the inventive implant, for example in a coating

In other exemplary embodiments, the therapeutically active agents, diagnostic and/or absorptive agents can be added by introducing them encapsulated, preferably encapsulated in polymeric shells, into the implant body. In these embodiments the agents represent the polymer particles and the encapsulating material is selected from materials as defined above for the biodegradable polymer particles that allow eluting of the active ingredients by partially or completely dissolving the encapsulating material in physiologic fluids.

Further functional modification can be achieved by adding, coating or partially or completely incorporating a material that alters and modulates, hereinafter referred to as altering and modulating material, the availability, function or release of a therapeutically active agent, diagnostic and/or absorptive agents. The altering and modulating material may comprise a diffusion barrier or a biodegradable material or a polymer or hydrogel. In some exemplary embodiments, the template or lumen may further comprise a combination of different active ingredients that are incorporated into different altering and modulating materials.

In other exemplary embodiments, the functional modification can be performed by application of a coating of one or more altering and modulating materials onto at least one part of the implant, whereby the polymer particles of the device comprise at least one therapeutically active agent, diagnostic or absorptive agent.

In further exemplary embodiments, it can be advantageous to coat the implant, or at least a part of the implant, with non-degradable or degradable polymers, optionally containing at least one active ingredient. Coatings controlling the release of active ingredients may also be used.

In another embodiment it can be desirable to coat the metallized template or the implant on the outer surface or inner surface with a coating to enhance engraftment or biocompatibility. Such coatings may comprise carbon coatings, metal carbides, metal nitrides, metal oxides e.g. diamond-like carbon or silicon carbide, or pure metal layers of e.g. titanium, using PVD, Sputter-, CVD or similar vapour deposition methods or ion implantation. In further exemplary embodiments a sol/gel-based coating that can be dissolvable in physiologic fluids may be applied to at least a part of the implant, as described in, e.g., International Publications WO 2006/077256 or WO 2006/082221.

Such coatings may be applied both to the metallized template as well as to the device after removal of the template or as a combination of both approaches.

In certain exemplary embodiments, it can be desirable to combine two or more different functional modifications as described above to obtain a functional implant.

Surface modification may be useful to provide a smooth surface. This may be done conventionally, for example by means of plasma treatment, polishing, electro polishing, sand-blasting, ion implantation, pitting and the like. It may further be desirable to add additional metal phases or metallic layers to the surface of the implant, for example by applying another metallization process that may be different from the one chosen for producing the metallized template.

In further exemplary embodiments, it may be desirable to produce a porous coating onto at least one part of the inventive implant in a further step, such as porous carbon coatings as described in International Publications WO 2004/101177, WO 2004/101017 or WO 2004/105826, or porous composite-coatings as described in International Publication WO 2006/082221 or International Patent Application PCT/EP2006/063450, or porous metal-based coatings as described in International Publications WO 2006/097503, or any other suitable porous coating. In this exemplary case, a further reservoir for the same or different active ingredients can be provided on the surface of the implant.

Having thus described in detail several exemplary embodiments of the present invention, it is to be understood that the present invention described above is not to be limited to particular details set forth in the above description, as many apparent variations thereof are possible without departing from the spirit or scope of the present invention. The exemplary embodiments of the present invention are disclosed herein or are obvious from and encompassed by the detailed description. The detailed description, given by way of example, is not intended to limit the present invention solely to the specific exemplary embodiments described herein.

The foregoing applications, and all documents cited therein or during their prosecution (“appln. cited documents”) and all documents cited or referenced in the appln. cited documents, and all documents cited or referenced herein (“herein cited documents”), and all documents cited or referenced in the herein cited documents, together with any manufacturer's instructions, descriptions, product specifications, and product sheets for any products mentioned herein or in any document incorporated by reference herein, are hereby incorporated herein by reference in their entireties, and may be employed in the practice of the present invention.

1. A process for manufacturing an implantable medical device or a part thereof, comprising:
i) providing a three-dimensional template of the device or of the part thereof;
ii) depositing at least one metallic layer covering the template; and
iii) at least partially removing the template.

2. The process of claim 1, further comprising generating at least one hollow space or lumen within a metallic structure defined by the at least one metallic layer.

3. The process of claim 1, further comprising generating a plurality of discrete lumens within a metallic structure defined by the at least one metallic layer.

4. The process of claim 1, wherein the at least one metallic layer substantially completely covers the template.

5. The process of claim 1, wherein the deposition of the metallic layer is performed in a one step operation.

6. The process of claim 1, wherein the at least one metallic layer is porous.

7. The process of claim 1, wherein the at least one metallic layer includes at least one opening before the at least partial removal of the template.

8. The process of claim 1, wherein the at least one metallic layer partially covers the template.

9. The process of claim 1, wherein the at least one metallic layer is deposited by at least one of a CVD procedure, a PVD procedure, an electroplating procedure, an electrodeposition procedure, an electroless plating procedure or a sol/gel precipitation procedure.

10. The process of claim 1, further comprising:
iv) forming at least one metallic implant structure that has within at least one hollow space; and
v) filling the at least one hollow space at least partly with an active ingredient.

11. The process of claim 1, wherein, after the at least partial removal of the template, the at least one metallic layer includes at least one opening.

12. The process of claim 1, wherein the template includes at least one of a polymeric material, an aerogel or an xerogel which can be removed in-vivo or ex-vivo.

13. The process of claim 1, wherein the template is composed of a material that is at least one of removable or degradable in-vivo.

14. The process of claim 1, wherein the template is composed of a material that is at least one of removable or degradable ex-vivo.

15. The process of claim 1, wherein the template is at least partially removed by at least one of (a) dissolving the template with particular solvents from a remaining hollow metallic structure of the implant, or (b) degrading the template thermally, mechanically or galvanically.

16. The process of claim 1, wherein the at least one metallic layer covering the template includes at least one of a metal, metal alloy or a biocompatible metallic material.

17. The process of claim 1, wherein the at least one metallic layer includes biodegradable metallic materials or an alloy comprising at least one of Mg, Ca, Fe, Zn, Al, W, Ln, Si, or Y.

18. The process of claim 17, wherein at least one of the biodegradable metallic materials include one of Mg or Zn.

19. A medical implant device, comprising:
at least one metallic layer having walls that enclose at least one lumen, and covering a three-dimensional template of the device or of the part thereof by being deposited thereon, wherein the template is at least partially removed.

20. The implant device of claim 19, further comprising at least one active ingredient.

21. The implant device of claim 20, wherein the at least one active ingredient comprises at least one of a pharmacologically, therapeutically or biologically active agent, a diagnostically active agent, a marker, or an absorptive agent

22. The implant device of claim 20, wherein the at least one active ingredient is included in the at least one lumen created by the at least partial removal of the template.

23. The implant device of claims 20, wherein the at least one active ingredient is included in at least one part of the at least one metallic layer.

24. The implant device of claim 20, wherein the at least one active ingredient is configured to be released from the at least one lumen of the implant.

25. The implant device of claim 19, further comprising at least one of a vascular endoprosthesis, an intraluminal endoprosthesis, a stent, a stent graft, a coronary stent, a peripheral stent, a surgical or orthopedic implant, an implantable orthopedic fixation aid, an orthopedic bone prosthesis or joint prosthesis, a bone substitute or bone graft or a vertebral substitute in the thoracic or lumbar region of the spinal column; an artificial heart or a part thereof, an artificial heart valve, a heart pacemaker casing or electrode, a subcutaneous and/or intramuscular implant, an implantable drug-delivery device, a microchip, or implantable surgical needles, screws, nails, clips, or staples.

26. The implant device of claim 20, wherein the at least one active ingredient is included in an in-vivo biodegradable template material for being releasable in-vivo through at least one opening or pore in the at least one metallic layer of the implant.

27. The implant device of claim 20, wherein the at least one active ingredient is included in an in-vivo biodegradable portion of the at least one metallic layer for being releasable in-vivo.
